SEQUENCE LISTING (106 sequences)

SEQ ID NO: 1 - DNA, 288 bp, Homo sapiens
  source 1..288: mol_type = genomic DNA
tactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 2 - DNA, 288 bp, Homo sapiens; identical to SEQ ID NO: 1
  source 1..288: mol_type = genomic DNA

SEQ ID NO: 3 - DNA, 392 bp, Homo sapiens
  source 1..392: mol_type = genomic DNA
aaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 4 - DNA, 393 bp, Homo sapiens
  source 1..393: mol_type = genomic DNA
taaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttc

SEQ ID NO: 5 - DNA, 247 bp, Homo sapiens
  source 1..247: mol_type = genomic DNA
ccacgccctctgtgaaagggcggggcatgcaaattcgaaatgaaagcccgggaacgccggaagaagcacgggtgtaagatttcccttttcaaaggcagagaataagaaatcagcccgagagtgtaagggcgtcaatagcgctgtggacgagacagagggaatggggcaaggagcgaggctggggctctcaccgcgacttgaatgtggatgagagtgggacggtgacggcgggcgcgaaggcgagcgc

SEQ ID NO: 6 - DNA, 250 bp, Homo sapiens
  source 1..250: mol_type = genomic DNA
ttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaatt

SEQ ID NO: 7 - DNA, 300 bp, Homo sapiens
  source 1..300: mol_type = genomic DNA
gcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggt

SEQ ID NO: 8 - DNA, 296 bp, Homo sapiens
  source 1..296: mol_type = genomic DNA
gtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 9 - DNA, 243 bp, Homo sapiens
  source 1..243: mol_type = genomic DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 10 - DNA, 106 bp, Homo sapiens
  source 1..106: mol_type = genomic DNA
caggactagtcttttaggtcaaaaagaagaagctttgtaaccgttggtttccgtagtgtagtggttatcacgttcgcctaacacgcgaaaggtccccggttcgaag

SEQ ID NO: 11 - DNA, 439 bp, Mus musculus
  source 1..439: mol_type = genomic DNA
ttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactc

SEQ ID NO: 12 - DNA, 258 bp, Mus musculus
  source 1..258: mol_type = genomic DNA
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 13 - DNA, 258 bp, Mus musculus; identical to SEQ ID NO: 12
  source 1..258: mol_type = genomic DNA

SEQ ID NO: 14 - DNA, 440 bp, Mus musculus
  source 1..440: mol_type = genomic DNA
tgccaacagctttgaaatcctctgtgctcttgtgccaatcccaccctcagaacagggagtctgtcggttatgttttcgtcctttacagttcttcgccgtctaatccattggaaacctcctttatttaaaggaaaccgtgtccaaattcagtgtacagaggtgcaaacctccgccccgctccttgtaggaagcccacgggaactgggccaatcggacggcgcttactcggctcatttacatacccataacacaccgcggctaatgcaaatattttcgtgaaaagatcttccattactcgaagttttaaaactaaaaacaaaatattatttcaaaagatcatgaagaaatttctgtcggtaacagcagtttcaattgataaatcaccatcacttatctaggaggttctgttactctagaagtgaattaagcaggacagctgt

SEQ ID NO: 15 - DNA, 231 bp, Mus musculus
  source 1..231: mol_type = genomic DNA
gggcggggcatgcaaataactgctctgtggaactctgggagcaaaaacaaaaaactgcaaccaaaacttctcggcctccttgaatcttacaggcttttcgtggcgtaaaggtggtgtactcaatgaagaggagagtctgtgttggctgcatgtttgagtcggttggttggtgactgtgaattaaaggtgtggtcggtgttgagtgtatggggcgtgtgggcgtagttcggt

SEQ ID NO: 16 - DNA, 311 bp, Mus musculus
  source 1..311: mol_type = genomic DNA
cgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgttt

SEQ ID NO: 17 - DNA, 97 bp, Homo sapiens
  source 1..97: mol_type = genomic DNA
catgcaaattacgcgctgtgctttgtgggaaatcaccctaaacgtaaaatttattcctctttcgagccttatagtggcggccggtctacaccctaaa

SEQ ID NO: 18 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 19 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 20 - DNA, 94 bp, Homo sapiens
  source 1..94: mol_type = genomic DNA
cctcttatgatgtttgttgccaatgatagattgttttcactgtgcaaaaattatgggtagttttggtggtcttgatgcagttgtaagcttggag

SEQ ID NO: 21 - DNA, 50 bp, Homo sapiens
  source 1..50: mol_type = genomic DNA
actttctggagtttcaaaaacagactgtacgccaagggtcatatcttttt

SEQ ID NO: 22 - DNA, 50 bp, Homo sapiens
  source 1..50: mol_type = genomic DNA
ccggggatacaacgtgtttcctaaaagtagagggaggtaagagacggtag

SEQ ID NO: 23 - DNA, 50 bp, Homo sapiens
  source 1..50: mol_type = genomic DNA
ctgaattttcttgcagttgaacaacagaggctttttttgtgtgtgtgggg

SEQ ID NO: 24 - DNA, 50 bp, Homo sapiens
  source 1..50: mol_type = genomic DNA
atatgtggtaatccaacaatagaaattatttttaagtttgtgtgttcctt

SEQ ID NO: 25-27 - skipped (3 entries)

SEQ ID NO: 28 - DNA, 206 bp, Mus musculus
  source 1..206: mol_type = genomic DNA
gtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagca

SEQ ID NO: 29 - DNA, 110 bp, Mus musculus
  source 1..110: mol_type = genomic DNA
cccaatttcactggtctacaatgaaagcaaaacagttctcttccccgctccccggtgtgtgagaggggctttgatccttctctggtttcctaggaaacgcgtatgtgtac

SEQ ID NO: 30 - DNA, 110 bp, Mus musculus; identical to SEQ ID NO: 29
  source 1..110: mol_type = genomic DNA

SEQ ID NO: 31 - DNA, 91 bp, Homo sapiens
  source 1..91: mol_type = genomic DNA
cttatgatgtttgttgccaatgatagattgttttcactgtgcaaaaattatgggtagttttggtggtcttgatgcagttgtaagcttggag

SEQ ID NO: 32 - DNA, 200 bp, Mus musculus
  source 1..200: mol_type = genomic DNA
ccacattttgtgtttaaaaatagaaatatttaagtgagatcagtttaaatctgctttatctagggtgtctaactgcttgcatctttttaacttttccttatttgtgagaaggtctgtaaaacttattatatgttagtacactgtagctgtcttcagacactccagaagagggattcagatcaccttacagatggttttga

SEQ ID NO: 33 - DNA, 78 bp, Mus musculus
  source 1..78: mol_type = genomic DNA
ccctctggggagtaaagttggttttaaagtcagagcatggtgattgtagggcagtccaacttttttaaatatgctgtg

SEQ ID NO: 34 - RNA, 101 nt, synthetic construct
  source 1..101: mol_type = other RNA
ggagttacctccaacatcaaggaagatggcagtaaccacaggttgtgtcaccagagtaacaaatttttggagtaggctttctggctccttaccggaaagcc

SEQ ID NO: 35 - RNA, 71 nt, synthetic construct
  source 1..71: mol_type = other RNA
ggagttacctccaacatcaaggaagatggcaaatttttggagtaggctttctggctccttaccggaaagcc

SEQ ID NO: 36 - RNA, 75 nt, synthetic construct
  source 1..75: mol_type = other RNA
ggagtgtaaccacaggttgtgtcaccagagtaacaaatttttggagtaggctttctggctccttaccggaaagcc

SEQ ID NO: 37 - RNA, 90 nt, synthetic construct
  source 1..90: mol_type = other RNA
ggagtcagcagcagcagcagcagcagcagcagcagcagcagcagcagcagaatttttggagtaggctttctggctccttaccggaaagcc

SEQ ID NO: 38 - RNA, 71 nt, synthetic construct
  source 1..71: mol_type = other RNA
ggagttcaccagaagcgtaccatactcacgaaatttttggagtaggctttctggctccttaccggaaagcc

SEQ ID NO: 39 - RNA, 98 nt, synthetic construct
  source 1..98: mol_type = other RNA
tacctccaacatcaaggaagatggcagtaaccacaggttgtgtcaccagagtaacaaatttttggagcaggttttctgacctccgtcggaaaacccct

SEQ ID NO: 40 - RNA, 70 nt, synthetic construct
  source 1..70: mol_type = other RNA
gtaaccacaggttgtgtcaccagagtaacaaatttttggagtaggctttctggctccttaccggaaagcc

SEQ ID NO: 41 - DNA, 2497 bp, synthetic construct
  source 1..2497: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttcaccagaagcgtaccatactcacgaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 42 - DNA, 4556 bp, synthetic construct
  source 1..4556: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtcagcagcagcagcagcagcagcagcagcagcagcagcagcagcagaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagactagtcgcgtcgacggatccgaattcgatatcgctagcataatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaagatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 43 - DNA, 3074 bp, synthetic construct
  source 1..3074: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttacctccaacatcaaggaagatggcagtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagctagtcgcgtaaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttacctccaacatcaaggaagatggcagtaaccacaggttgtgtcaccagagtaacaaatttttggagtaggctttctggctccttaccggaaagccactttctggagtttcaaaaacagactgtacgccaagggtcatatctttttcgtcgacggatccgcaaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgtttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaacccatgcagagtacctctagagaaaagtttctaaagttggttttgccttgcttagaaaggaggtcaaaggtgaagcctaggagaaacattcagcaatgttgcacaaagacatgagaataaatgattttagcatcctgaaatgaagagcatagtgatctcaccatagctacccacctacccagagaacttccaggctttcactgtctcttttctgaatcacccacattacagaatatcagtcattgtgtaattagataacagattgtgaggtgcccaaacagaaacaaactttacaggaggagagagaaaatcaaggtgatgactatggagaacaaaatgagaaggagaggtggagttcctgctgcatgaacacctatcctgctatagagcccatttcttccataaataatacaactctgtaactccataggtcagaaagaaacagtgttctgtgtcttcccatctagcaccacaaacccccacaagattaatttgtttctagggacccttaaatctctatcaaaattctggaaacctctactttagaaaatcttatatttattattcaggctactttccagagtgataagctactgagtctcctaagtgtcatctatggtacacagggataagatcaggaataaacctgggaattcgatatctagatctcgaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 2383 bp, synthetic construct
  source 1..2383: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaaccggtcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttacctccaacatcaaggaagatggcaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagcaaactacagagccaagtgctatccacagacttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtgtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggagctttctgggttgccatctcaagcagaccttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtgtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaagtacaaggccatcagctcatactcacaattgcttcgcgatgtacgggccagatatatactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttacctccaacatcaaggaagatggcaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtgcggacccaacggccgcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 45 - DNA, 2210 bp, synthetic construct
  source 1..2210: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaataaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttacctccaacatcaaggaagatggcagtaaccacaggttgtgtcaccagagtaacaaatttttggagtaggctttctggctccttaccggaaagccactttctggagtttcaaaaacagactgtacgccaagggtcatatctttttttgtattggtttgtgtcttggttggtgtcttactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagttacctccaacatcaaggaagatggcagtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggagttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaattggagttacctccaacatcaaggaagatggcagtaaccacaggttgtgtcaccagagtaacaaatttttggagtaggctttctggctccttaccggaaagccctgaattttcttgcagttgaacaacagaggctttttttgtgtgtgtgggggtgcttggttttgggaggttgaagagtactgcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggtggagttacctccaacatcaaggaagatggcaaatttttggagtaggctttctggctccttaccggaaagccatatgtggtaatccaacaatagaaattatttttaagtttgtgtgttcctttttctgttcaatggtgcttttgatattgttgcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 46 - DNA, 2128 bp, synthetic construct
  source 1..2128: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccggtcgcgtctagtaataaggaccagcttctttgggagagaacagacgcaggggcgggagggaaaaagggagaggcagacgtcacttccccttggcggctctggcagcagattggtcggttgagtggcagaaaggcagacggggactgggcaaggcactgtcggtgacatcacggacagggcgacttctatgtagatgaggcagcgcagaggctgacgtcttcgccacttgctgcttcaccacgaaggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagaggcacaacgtttcggagttacctccaacatcaaggaagatggcaaatttttggagtaggctttctggctccttaccggaaagccactttctggagtttcaaaaacagactgtacgccaagggtcatatctttttttgtattggtttgtgtcttggttggtgtcttactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtgtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggagttcgcagtctctgaattaagtctattagcatgttcctcccatagtgctttgcttcatatcaacaaaaacctagctaagtgaaatcagcaacgatatgcagaaaccacctacgcaggtcacaaacatctttctatgattgtataattttcaagcaagcaataagtgaagatttttccataggccctaaactcacctttgcgaaataggaagctggtttattgggagtgatgagcagggggcgtaacaaattggagtgtaaccacaggttgtgtcaccagagtaacaaatttttggagtaggctttctggctccttaccggaaagccctgaattttcttgcagttgaacaacagaggctttttttgtgtgtgtgggggtgcttggttttgggaggttgaagagtactgcagcaaggcctccacttcaccccctaaaggttgccccaagagcaccgtgtgactgctaaggtatttccggagtctaaagacgattattcaggtctcatttgcatacccataatacactgcaaacagtatttttttcggaaaaacatttatatattgcttgacatttttaagtatgagaattttgcatgcagaatttttttgtataaactttctcaggtagtaacccttgggattagtagacaccatcagtgtactaggaattgcagttacccgaaaattgagttacagaagtaactggtggagttacctccaacatcaaggaagatggcaaatttttggagtaggctttctggctccttaccggaaagccatatgtggtaatccaacaatagaaattatttttaagtttgtgtgttcctttttctgttcaatggtgcttttgatattgttgcctagtccactccctctctgcgcgctcgctcgctcactgaggccgggcgaccaaaggtcgcccgacgcccgggctttgcccgggcggcctcagtgagcgagcgagcgcgcagagaggga

SEQ ID NO: 47 - DNA, 2210 bp, synthetic construct; identical to SEQ ID NO: 45
  source 1..2210: mol_type = other DNA

SEQ ID NO: 48 - DNA, 1276 bp, synthetic construct
  source 1..1276: mol_type = other DNA
ctgcgcgctcgctcgctcactgaggccgcccgggcaaagcccgggcgtcgggcgacctttggtcgcccggcctcagtgagcgagcgagcgcgcagagagggagtggggttacttcgcgatgtacgggccagatatatactgccgtaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttacctccaacatcaaggaagatggcaaatttttggagtaggctttctggctccttaccggaaagcccccaatttcactggtctacaatgaaagcaaaacagttctcttccccgctccccggtgtgtgagaggggctttgatccttctctggtttcctaggaaacgcgtatgtgtaccgtcgacggatccgaattcgatatctagatctcgaggtaaccacgtgtactgccgaatccaggtctccgggcttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcaggagtgtaaccacaggttgtgtcaccagagtaacaaatttttggagtaggctttctggctccttaccggaaagcccctcttatgatgtttgttgccaatgatagattgttttcactgtgcaaaaattatgggtagttttggtggtcttgatgcagttgtaagcttggaggatccgagctcggtaccaaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagc

SEQ ID NO: 49 - DNA, 1532 bp, synthetic construct
  source 1..1532: mol_type = other DNA
ctgcgcgctcgctcgctcactgaggccgcccgggcaaagcccgggcgtcgggcgacctttggtcgcccggcctcagtgagcgagcgagcgcgcagagagggagtggggttcttcgaaacaccggttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaaggagttacctccaacatcaaggaagatggcagtaaccacaggttgtgtcaccagagtaacaaatttttggagcaggttttctgacctccgtcggaaaacccctcccaatttcactggtctacaatgaaagcaaaacagttctcttccccgctccccggtgtgtgagaggggctttgatccttctctggtttcctaggaaacgcgtatgtgttgttcctcttagtgttaattcacactaaagactgtgcatccgactcctacatttatgaaagtaaatgcctgttgttagaacaaaaaaggctacagaacaaaaaacaaagcgaaataccatctgctttaggttcagtgtggtattttcccgctgacagggaggcgggtttttgggtacaggaaacgagtcactatggaggcggtactatgtagatgagaattcaggtgcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcggagttacctccaacatcaaggaagatggcagtaaccacaggttgtgtcaccagagtaacaaatttttggagcaggttttctgacctccgtcggaaaacccctgtttacttggttttaaaaatagcttgcactagcgatacggaatatggttattaggtttgttaggcatcatgtcgtgtcttactatagaaaaataacgtagtgttcattttagcctgcctgtatgtgttaatttgtccttattgcgcattgttcttgttaagtcttctgtaaggagttgcgggtttcaaactgtcagtctgagagcagaattcgatatctagatctcgaggtaaccacgtgcggacccaacggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 50 - DNA, 110 bp, synthetic construct
  source 1..110: mol_type = other DNA
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 51 - DNA, 130 bp, synthetic construct
  source 1..130: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 52 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 53 - DNA, 113 bp, synthetic construct
  source 1..113: mol_type = other DNA
ccactccctctctgcgcgctcgctcgctcactgaggccgggcgaccaaaggtcgcccgacgcccgggctttgcccgggcggcctcagtgagcgagcgagcgcgcagagaggga

SEQ ID NO: 54 - DNA, 133 bp, synthetic construct
  source 1..133: mol_type = other DNA
aaggaacccctagtgatggagttggccactccctctctgcgcgctcgctcgctcactgaggccgggcgaccaaaggtcgcccgacgcccgggctttgcccgggcggcctcagtgagcgagcgagcgcgccagc

SEQ ID NO: 55 - DNA, 141 bp, synthetic construct; identical to SEQ ID NO: 52
  source 1..141: mol_type = other DNA

SEQ ID NO: 56 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 57 - RNA, 27 nt, Homo sapiens
  source 1..27: mol_type = genomic RNA
ggctttctggctccttaccggaaagcc

SEQ ID NO: 58 - RNA, 27 nt, Homo sapiens
  source 1..27: mol_type = genomic RNA
ggctttctgggaggttaccggaaagcc

SEQ ID NO: 59 - RNA, 28 nt, Homo sapiens
  source 1..28: mol_type = genomic RNA
ggctttctggcctccttaccggaaagcc

SEQ ID NO: 60 - RNA, 28 nt, Homo sapiens
  source 1..28: mol_type = genomic RNA
ggctttctggggaggttaccggaaagcc

SEQ ID NO: 61 - RNA, 27 nt, Homo sapiens
  source 1..27: mol_type = genomic RNA
ggctttctggctggctaccggaaagcc

SEQ ID NO: 62 - RNA, 25 nt, Homo sapiens
  source 1..25: mol_type = genomic RNA
ggctttctggcttccccggaaagcc

SEQ ID NO: 63 - RNA, 27 nt, Homo sapiens
  source 1..27: mol_type = genomic RNA
ggctttctggcttcttcccggaaagcc

SEQ ID NO: 64 - RNA, 27 nt, Homo sapiens
  source 1..27: mol_type = genomic RNA
ggctttctggcaacttaccggaaagcc

SEQ ID NO: 65 - RNA, 27 nt, Homo sapiens
  source 1..27: mol_type = genomic RNA
ggctttctggttcggtaccggaaagcc

SEQ ID NO: 66 - RNA, 28 nt, Homo sapiens
  source 1..28: mol_type = genomic RNA
ggctttctggaagccttaccggaaagcc

SEQ ID NO: 67 - RNA, 27 nt, Homo sapiens
  source 1..27: mol_type = genomic RNA
ggctttctggcttcttaccggaaagcc

SEQ ID NO: 68 - RNA, 29 nt, Homo sapiens
  source 1..29: mol_type = genomic RNA
ggctttctggcctccgccggaaagcccct

SEQ ID NO: 69 - RNA, 30 nt, Mus musculus
  source 1..30: mol_type = genomic RNA
ggctttctggctccttaccggaaagcccct

SEQ ID NO: 70 - RNA, 29 nt, Mus musculus
  source 1..29: mol_type = genomic RNA
ggttttctgacctccgtcggaaaacccct

SEQ ID NO: 71 - RNA, 33 nt, Mus musculus
  source 1..33: mol_type = genomic RNA
ggttttctgacctccttcggtcggaaaacccct

SEQ ID NO: 72 - RNA, 26 nt, Mus musculus
  source 1..26: mol_type = genomic RNA
ggttttctgacctccgtcggaaaacc

SEQ ID NO: 73 - RNA, 30 nt, Mus musculus
  source 1..30: mol_type = genomic RNA
ggttttctgacactccgtcggaaaacccct

SEQ ID NO: 74 - RNA, 29 nt, Mus musculus
  source 1..29: mol_type = genomic RNA
ggttttctgatctccatcggaaaacccct

SEQ ID NO: 75 - RNA, 29 nt, Mus musculus
  source 1..29: mol_type = genomic RNA
ggttttccgacctccgtcggaaaacccct

SEQ ID NO: 76 - RNA, 30 nt, synthetic construct
  source 1..30: mol_type = other RNA
ggctttctggcactccaccggaaagcccct

SEQ ID NO: 77 - RNA, 30 nt, synthetic construct
  source 1..30: mol_type = other RNA
ggctttctggcactccgccggaaagcccct

SEQ ID NO: 78 - RNA, 29 nt, synthetic construct
  source 1..29: mol_type = other RNA
ggctttctggcctccaccggaaagcccct

SEQ ID NO: 79 - RNA, 29 nt, synthetic construct
  source 1..29: mol_type = other RNA
ggttttccggtctccaccggaaagccccc

SEQ ID NO: 80 - RNA, 29 nt, synthetic construct
  source 1..29: mol_type = other RNA
ggctttccggtctccaccggaaagcccct

SEQ ID NO: 81 - RNA, 29 nt, synthetic construct
  source 1..29: mol_type = other RNA
ggctttccggcctccgccggaaagcccct

SEQ ID NO: 82 - RNA, 29 nt, synthetic construct
  source 1..29: mol_type = other RNA
ggttttccggtctccaccggaaaaccctt

SEQ ID NO: 83 - RNA, 29 nt, synthetic construct
  source 1..29: mol_type = other RNA
ggttttccgtgctcccacggaaaaccctt

SEQ ID NO: 84 - RNA, 29 nt, synthetic construct
  source 1..29: mol_type = other RNA
ggttttccggcctccgccggaaaaccctt

SEQ ID NO: 85 - RNA, 30 nt, synthetic construct
  source 1..30: mol_type = other RNA
ggttttccgtgactcccacggaaaaccctt

SEQ ID NO: 86 - RNA, 30 nt, synthetic construct
  source 1..30: mol_type = other RNA
ggttttccggcactccgccggaaaaccctt

SEQ ID NO: 87 - RNA, 29 nt, synthetic construct
  source 1..29: mol_type = other RNA
ggtcttccggtctcctccggaaggccccc

SEQ ID NO: 88 - RNA, 27 nt, synthetic construct
  source 1..27: mol_type = other RNA
ggtcttccggctccccggaaggccccc

SEQ ID NO: 89 - RNA, 29 nt, synthetic construct
  source 1..29: mol_type = other RNA
ggctttccgtgctcccacggaaagcccct

SEQ ID NO: 90 - RNA, 30 nt, synthetic construct
  source 1..30: mol_type = other RNA
ggctttccgtgactcccacggaaagcccct

SEQ ID NO: 91 - RNA, 30 nt, synthetic construct
  source 1..30: mol_type = other RNA
ggctttccggcactccgccggaaagcccct

SEQ ID NO: 92 - RNA, 29 nt, Homo sapiens
  source 1..29: mol_type = genomic RNA
ggttttctgacttcggtcggaaaacccct

SEQ ID NO: 93 - RNA, 26 nt, Homo sapiens
  source 1..26: mol_type = genomic RNA
ggttttctgacttcggtcggaaaacc

SEQ ID NO: 94 - RNA, 27 nt, Homo sapiens
  source 1..27: mol_type = genomic RNA
ggctttctggctttttaccggaaagcc

SEQ ID NO: 95 - RNA, 30 nt, Homo sapiens
  source 1..30: mol_type = genomic RNA
ggctttctggctttttaccggaaagcccct

SEQ ID NO: 96 - RNA, 29 nt, Homo sapiens
  source 1..29: mol_type = genomic RNA
ggctttccggcctccgccggaaagcccct

SEQ ID NO: 97 - RNA, 26 nt, Homo sapiens
  source 1..26: mol_type = genomic RNA
ggctttccggcctccgccggaaagcc

SEQ ID NO: 98 - DNA, 30 bp, synthetic construct
  source 1..30: mol_type = other DNA
caaactacagagccaagtgctatccacaga

SEQ ID NO: 99 - DNA, 30 bp, synthetic construct
  source 1..30: mol_type = other DNA
gagctttctgggttgccatctcaagcagac

SEQ ID NO: 100 - DNA, 30 bp, synthetic construct
  source 1..30: mol_type = other DNA
tacaaggccatcagctcatactcacaattg

SEQ ID NO: 101 - DNA, 100 bp, synthetic construct
  source 1..100: mol_type = other DNA
caaactacagagccaagtgctatccacagagagctttctgggttgccatctcaagcagactacaaggccatcagctcatactcacaattgactttgagag

SEQ ID NO: 102 - DNA, 100 bp, synthetic construct
  source 1..100: mol_type = other DNA
ttgaccacatacgtgctctttcaaagttctgtgtttgaagttatgttagtaacaactgatgcccatcctgcaatgacaaatccaattctcagtgcagctc

SEQ ID NO: 103 - DNA, 500 bp, synthetic construct
  source 1..500: mol_type = other DNA
caaactacagagccaagtgctatccacagagagctttctgggttgccatctcaagcagactacaaggccatcagctcatactcacaattgactttgagagtcattttccaatgctcctacacaccccttcttcacaatccccaacaaatctgaggctggaacttggtaccataacaatcattacattatttcaccagaagtacaccttgcctggaagattggcattatagcatcttctaacattgtgaaagttagtgaccaatgaggagatccaagtcagttccagttggatttctctatactctataataaatatatatggtgtcttcaacaataggactttgccatccagtgatgctaaaaatcaataacaatggcaataacctgccctgtttggaaagcctctggcttccatgactaacaattcaaggcaggtctcctatacctagtactgagatttttatttgataaactatatcttctgggaggagaagcattgt

SEQ ID NO: 104 - DNA, 500 bp, synthetic construct
  source 1..500: mol_type = other DNA
ttgaccacatacgtgctctttcaaagttctgtgtttgaagttatgttagtaacaactgatgcccatcctgcaatgacaaatccaattctcagtgcagctctctgaaatagttttgctttctctctctaggtctgttctatactcctaactctccaggagtttacaaggaataaaatctcttccaaatgctttctgttgcaacaactggaccatactgaaagctgaggcccacaattgcaatctaggttagcaggtaatcattgttggtgaggtcctccctttccccaggctcgtgtttgtattggggagcaggaaatttttgctagagcagcactgccatctctctacactccacctgattggtgggatggaccagagaaatggacattcccaacacagtccctcctttcacatctgctcacctgcccacaggatactttccaccatgcatactgggctctgcaccaaccattcagcagtgatgaagaggaaacttgaac

SEQ ID NO: 105 - RNA, 13 nt, synthetic construct
  source 1..13: mol_type = other RNA
aatttttggagca

SEQ ID NO: 106 - RNA, 13 nt, synthetic construct
  source 1..13: mol_type = other RNA
aatttttggagta

(19) United States
(12) Patent Application Publication
(10) Pub. No.: US 2026/0201412 A1
(43) Pub. Date: Jul. 16, 2026

(54) ADENO-ASSOCIATED VIRAL VECTORS FOR PROPER PACKAGING OF REPETITIVE ELEMENTS

(71) Applicant: Regeneron Pharmaceuticals, Inc., Tarrytown, NY (US)

(72) Inventors: Gregory Thomas NACHTRAB, San Diego, CA (US); Bret D. REID, San Diego, CA (US); Ranjan BATRA, San Diego, CA (US); Rea LARDELLI MARKMILLER, San Diego, CA (US); Rachel A. ADAMS, San Diego, CA (US)

(21) Appl. No.: 19/134,044

(22) PCT Filed: Dec. 1, 2023
(86) PCT No.: PCT/US2023/082124

§ 371 Date: May 29, 2025

Related U.S. Application Data
(60) Provisional application No. 63/385,759, filed on Dec. 1, 2022.

(51) Int. Cl. C12N 15/86; A61K 48/00; C12N 15/113

(52) U.S. Cl. CPC C12N 15/86; C12N 15/113; A61K 48/00; C12N 2750/14143

(57) ABSTRACT

Disclosed are improved vectors for packaging and/or expressing multiple noncoding RNAs or protein coding transgenes each governed by distinct promoter sequences.

### CROSS REFERENCE TO RELATED APPLICATIONS

This application claims priority to, and the benefit of, U.S. provisional application Nos. 63/385,759, filed Dec. 1, 2022 the contents of which are hereby incorporated by reference in its entirety.

### FIELD OF THE DISCLOSURE

The disclosure is directed to molecular biology, gene therapy, and compositions and methods for modifying expression and activity of RNA molecules.

### REFERENCE TO AN ELECTRONIC SEQUENCE LISTING

The contents of the electronic sequence listing (LOCN_023_001WO_SeqList_ST26.xml; Size: 123,085 bytes; and Date of Creation: Nov. 30, 2023) is herein incorporated by reference in its entirety.

### BACKGROUND

There are long-felt but unmet needs in the art for providing effective therapies for correcting dysfunctional messenger RNA.

Recombinant AAV is one of the delivery vehicles of choice for gene therapy. Emerging programmable technologies are now compact enough to allow for the targeting of multiple genes at that DNA or RNA level in a single AAV vector via the packaging of multiple targeting nucleic acid molecules such as guide RNA (gRNA) or small-nuclear RNA (snRNA). However, these targeting molecules often share the same regulatory sequences. Homology between the shared regulatory sequences drives undesirable truncations and deletions in recombinant AAV genomes. Reducing and/or eliminating this sequence homology eliminating these truncations/deletions, allowing for complete genome packaging, and higher levels of vector production (i.e., higher titers) of the packaged targeting nucleic acid molecules.

Accordingly, the disclosure provides compositions and methods comprising a new therapeutic RNA-targeting platform comprised of engineered snRNAs.

### SUMMARY

The disclosure provides a recombinant Adeno-Associated Virus (rAAV) vector comprising: a first AAV inverted terminal repeat (ITR) sequence, a first promoter sequence, a first nucleic acid element, a second promoter sequence, a second nucleic acid element, and a second ITR sequence, wherein the first promoter sequence and second promoter sequence are distinct promoter sequences.

In some aspects, the first nucleic acid element and second nucleic acid element comprise a nucleic acid encoding a noncoding RNA, or a transgene.

In some aspects, the noncoding RNA is a small-nuclear RNA (snRNA) molecule, a single guide RNA molecule (sgRNA), a microRNA, a short hairpin RNA (shRNA), an enhancer RNA (eRNA), a small nucleolar RNA (snoRNA), or a long noncoding RNA (lncRNA).

In some aspects, the rAAV vector further comprising one or more additional promoter sequences.

In some aspects, the one or more additional promoter sequences are distinct from the first promoter sequence and the second promoter sequence.

In some aspects, the rAAV vector further comprising one or more additional nucleic acid elements.

In some aspects, the first promoter sequence has less than 75% sequence identity to the second promoter sequence and the one or more additional promoter sequences.

In some aspects, there are from about 50 to about 5,000 nucleotides between the 3′ end of the first promoter and the 5′ start of the second promoter.

In some aspects, the length of each of the first nucleic acid elements, the second nucleic acid element, and the one or more additional nucleic acid elements is between about 50 and 5,000 nucleotides.

In some aspects, the first nucleic acid element, second nucleic acid element, and/or one ore more additional nucleic acid elements comprise one snRNA molecule.

In some aspects, the first nucleic acid element, second nucleic acid element, and/or one or more additional nucleic acid elements comprises two snRNA molecules.

In some aspects, the two snRNA molecules are separated by a spacer sequence site. In some aspects, the snRNA molecule is a modified snRNA molecule.

In some aspects, the snRNA molecule comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 34-SEQ ID NO: 40.

In some aspects, the promoter is selected from a U1, U2, U4, U5, U6, U7, H1, 7SK, or tRNA promoter.

In some aspects, the promoter controls expression of an mRNA-encoding gene.

In some aspects, the U1 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 3, SEQ ID NO: 4 or SEQ ID NO: 11.

In some aspects, the U4 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 6.

In some aspects, the U5 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 7.

In some aspects, the U7 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 1, SEQ ID NO: 2, SEQ ID NO: 12, or SEQ ID NO: 13.

In some aspects, the first ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 50-SEQ ID NO: 55.

In some aspects, the second ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 50-SEQ ID NO: 55.

In some aspects, the rAAV vector further comprising one or more terminator sequences. In some aspects, the terminator sequence is a U1, U2, U4, U5, U6, or U7 terminator sequence. In some aspects, the terminator sequence is a polyA sequence or a PolIII termination sequence.

In some aspects, the U1 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO:21 or SEQ ID NO: 28.

In some aspects, the U4 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 23.

In some aspects, the U5 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 24 or SEQ ID NO: 32.

In some aspects, the U7 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 20, SEQ ID NO: 29, or SEQ ID NO: 30.

In some aspects, the rAAV vector is a single-stranded AAV vector (ssAAV). In some aspects, the rAAV vector is a self-complementary AAV vector (scAAV).

The disclosure provides an AAV viral vector comprising the rAAV vector of any one of the preceding embodiments wherein the viral vector comprises an AAV capsid protein.

In some aspects, the AAV capsid protein is an AAV1 capsid protein, an AAV2 capsid protein, an AAV3 capsid protein, an AAV3B capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein, an AAVrh10 capsid protein, or a modified AAV capsid protein.

In some aspects, the AAV viral vector exhibits greater expression in a subject or cell relative to an AAV viral vector comprising an rAAV vector comprising a single noncoding RNA or transgene or an rAAV vector comprising repeated promoter sequences operably linked to noncoding RNA molecules or transgene sequences.

The disclosure provides a pharmaceutical composition comprising the AAV viral vector of any embodiment disclosed herein.

The disclosure provides a cell comprising the rAAV vector or the AAV viral vector of any embodiment disclosed herein.

The disclosure provides a method of treating a disease or disorder in a subject in need thereof comprising administering a therapeutically effective amount of the AAV viral vector of claim 29 or the pharmaceutical composition of any embodiment disclosed herein.

### BRIEF DESCRIPTION OF THE DRAWINGS

FIG. 1A is a schematic depicting various snRNA expression cassettes comprised of promoters and snRNA molecules in various permutations. A02888 depicts a single-stranded AAV (ssAAV) snRNA expression cassette comprising a single CUG repeat targeting snRNA controlled by a U7 promoter. A02896 depicts an ssAAV snRNA expression cassette comprising a three CUG repeat targeting snRNA molecules each under the control of a U7 promoter. A03624 depicts a self-complementary AAV (scAAV) snRNA expression cassette comprising from 5′ to 3′: a U7 promoter driving expression of snRNA 38, a U7 promoter driving expression of snRNA 42, a U7 promoter driving expression of snRNA 42, and a U7 promoter driving expression of snRNA 38. A03081 depicts an scAAV snRNA expression cassette comprising from 5′ to 3′: a U7 promoter driving expression of a CAG-targeting snRNA, a U7 promoter driving expression of a CAG-targeting snRNA, a U7 promoter driving expression of a CAG-targeting snRNA, and a U7 promoter driving expression of a CAG-targeting snRNA.

FIG. 1B is a series of tapestation images depicting rAAV vectors comprising the snRNA expression cassettes of FIG. 1A. 1X refers to an AAV vector with 1 expression cassette (i.e. one promoter operably linked to one snRNA). 2X refers to refers to an AAV vector with 2 expression cassettes. 3X refers to refers to an AAV vector with 3 expression cassettes. 4X refers to refers to an AAV vector with 4 expression cassettes.

FIG. 1C is a graph depicting the full-length sequencing of an AAV9 viral vector comprising the A03624 snRNA expression cassette. The y-axis depicts downsampled read # and the x-axis depicts read length.

FIG. 2 is a series of schematics of rAAV vectors comprising snRNA expression cassettes having repeats of the same three promoters. Depicted are interactions and/or annealing of complementary regions, for example the repeated U7 promoters, of two rAAV vectors. Depicted are ssAAV and scAAV vectors.

FIG. 3 is a schematic depicting various snRNA expression cassettes comprised of promoters and snRNA molecules in various permutations. Two ssAAV snRNA expression cassettes are depicted, the first depicting the 38/42 snRNA molecules under the control of a U7 promoter and a second 38/42 snRNA under control of a U1 promoter. A second expression cassette is depicted that further comprises a stuffer sequence. Two scAAV snRNA expression cassettes are depicted, the first depicting snRNA molecule, 38/42 under the control of a U7 promoter and a second snRNA 38/42 under control of a U1 promoter. The second scAAV expression cassette depicts from 5′ to 3′: a U1 promoter driving expression of the 38/42 snRNA molecule, a U7 promoter driving expression of the 38/42 snRNA molecule, a U4 promoter driving expression of the 38/42 snRNA molecule, and a U5 promoter driving expression of the 38/42 snRNA molecule.

FIG. 4A is a schematic depicting ssAAV snRNA expression cassettes A03981 and A04184. A03981 comprises snRNA molecule, 38 under the control of a U7 promoter and snRNA 42 under control of a U1 promoter. A04184 depicts the 38/42 snRNA molecule under the control of a U7 promoter and a second 38/42 snRNA molecule under control of a U1 promoter as well as a stuffer sequence.

FIG. 4B is a series of tapestation images of AAV vectors comprising A03981 and A04184.

FIG. 4C is a graph depicting the full-length sequencing of an AAV9 viral vector comprising the A03981 snRNA expression cassette. The y-axis depicts downsampled read # and the x-axis depicts read length. Single stranded and dimer/self-complementary (SC) peaks are annotated.

FIG. 4D is a graph depicting the full-length sequencing of an AAV9 viral vector comprising the A04184 snRNA expression cassette. The y-axis depicts downsampled read # and the x-axis depicts read length.

FIG. 4E is a visualized of the sequencing coverage of the plasmid (pAAV vector) containing the genome for A04184. The sequencing reads almost completely map to the intended genome ITRs/expression cassette.

FIG. 5A is a schematic depicting scAAV snRNA expression cassettes A04232 and A04234. A04232 comprises the 38/42 snRNA molecule under the control of a U7 promoter and the 38/42 snRNA molecule under control of a U1 promoter. A04234 depicts an snRNA molecule targeting CUG repeats under the control of a U7 promoter and a second pair snRNA targeting CUG repeats under control of a U1 promoter.

FIG. 5B is a series of tapestation images of AAV vectors comprising A04232 and A04234.

FIG. 5C is a graph depicting the full-length sequencing of an AAV9 viral vector comprising the A04232 snRNA expression cassette. The y-axis depicts downsampled read # and the x-axis depicts read length. Single stranded and self-complementary (SC) peaks are annotated.

FIG. 5D is a visualized of the sequencing coverage of the plasmid (pAAV vector) containing the genome for A04232. The sequencing reads in the shorter “ssAAV bin” almost completely map to the intended genome ITRs/expression cassette. This indicates these are full length ssAAV genomes and not unintended partial genomes or truncations.

FIG. 5E is a visualized of the sequencing coverage of the plasmid (pAAV vector) containing the genome for A04232. The sequencing reads in the shorter “ssAAV bin” almost completely map to the intended genome ITRs/expression cassette.

FIG. 6A is a schematic depicting scAAV snRNA expression cassettes A03624 and A04226. A03624 depicts a self-complementary AAV (scAAV) snRNA expression cassette comprising from 5′ to 3′: a U7 promoter driving expression of snRNA 38, a U7 promoter driving expression of snRNA 42, a U7 promoter driving expression of snRNA 42, and a U7 promoter driving expression of snRNA 38. A04226 depicts from 5′ to 3′: a U1 promoter driving expression of snRNA 38/42, a U7 promoter driving expression of snRNA 38/42, a U4 promoter driving expression of snRNA 38/42, and a U5 promoter driving expression of snRNA 38/42.

FIG. 6B is a series of tapestation images of AAV vectors comprising A03624 and A04226.

FIG. 6C is the tapestation sample trace of the A03624 shown in FIG. 6B.

FIG. 6D is the tapestation sample trace of the A04226 shown in FIG. 6B.

FIG. 7A is a series of tapestation images depicting AAV vectors comprising snRNA expression cassettes comprising various promoter permutations. “U7” refers to an snRNA expression cassette wherein a single U7 promoter driving expression of at least one snRNA molecule. “U7 U7 U7” refers to an snRNA expression cassette wherein three U7 promoters each drive expression of at least one snRNA molecule. “U1 U7” refers to an snRNA expression cassette wherein one U1 promoter drives expression of at least one snRNA molecule and one U7 promoter drives expression of at least one snRNA molecule.

FIG. 7B is a series of graphs depicting the full-length sequencing of AAV9 viral vectors comprising the snRNA expression cassettes from FIG. 7A. The y-axis depicts downsampled read # and the x-axis depicts read length.

FIG. 8A is a series of tapestation images depicting AAV vectors comprising snRNA expression cassettes comprising various promoter permutations. “U7 U7 U7 U7” refers to an snRNA expression cassette wherein four U7 promoters each drive expression of at least one snRNA molecule. “U1 U7 U4 U5 same snRNA” refers to an snRNA expression cassette wherein a U1 promoter, a U7 promoter, a U4 promoter, and a U5 promoter each drive expression of a discrete copy of an snRNA molecule such that 4 copies of the snRNA molecule are present in the cassette. “U1 U7 U4 U5 different snRNA” refers to an snRNA expression cassette wherein a U1 promoter, a U7 promoter, a U4 promoter, and a U5 promoter each drive expression of a different snRNA molecule such that the expression cassette comprises 4 snRNA molecules having distinct sequences.

FIG. 8B is the tapestation sample trace and quantification of the samples shown in FIG. 8A.

FIG. 8C is a series of graphs depicting the full-length sequencing of AAV9 viral vectors comprising the “U1 U7 U4 U5 same snRNA” and “U1 U7 U4 U5 different snRNA” snRNA expression cassettes from FIG. 8A. The y-axis depicts downsampled read # and the x-axis depicts read length

FIG. 9 is a graph depicting copies of AAV vector per ng of RNA for a variety of AAV viral vectors comprising snRNA expression cassettes. Evaluated snRNA expression cassettes include: a) 4×U7 (four U7 promoters each driving expression of the same snRNA molecule at two different MOIs 3e5 and 1e6; b) U1 and U7 promoters each driving expression of an snRNA molecule at three MOIs 1e5, 5e5, and 1e6; c) U1, U7, U4 and U5 promoters each driving expression of an snRNA molecule at three MOI2 1e5, 5e5, and 1e6.

FIG. 10A is a schematic depicting various snRNA expression cassettes comprised of promoters and snRNA molecules in various permutations. A04384 depicts a scAAV snRNA expression cassette comprising snRNA 38 controlled by a murine U7 promoter and snRNA 42 controlled by a human U7 promoter.

FIG. 10B is a tapestation images depicting rAAV vectors comprising the snRNA expression cassettes of FIG. 10A.

FIG. 10C is a tapestation trace of the tapestation image of FIG. 10B.

FIG. 10D is an alignment of the mouse and human U7 promoter depicting 57.3% sequence similarity and 57.3% sequence identity.

FIG. 11A is a schematic depicting various snRNA expression cassettes comprised of promoters and snRNA molecules in various permutations. A04526 depicts a scAAV snRNA expression cassette comprising snRNA 38/42 controlled by a murine U7 promoter and snRNA 38/42 controlled by a human U7 promoter.

FIG. 11B is a tapestation images depicting rAAV vectors comprising the snRNA expression cassettes of FIG. 11A.

FIG. 11C is a tapestation trace of the tapestation image of FIG. 10B.

### DETAILED DESCRIPTION

Traditional designs for expressing multiple snRNA or sgRNA use the same promoter (often U7 or U6) several times in the AAV genome. Through genome extraction and sequencing analysis, it was observed that these repetitive genomes undergo significant truncations or deletions during AAV packaging. Reduction of repetitive sequences in the genome by using different promoters and/or snRNA sequences enables a correction of AAV genome aberrations leading to improved AAV packaging and increased snRNA expression.

The disclosure provides expression vectors, including recombinant Adeno-Associated Virus (rAAV) vectors, comprising one or more nucleic acid elements each operably linked to a promoter wherein the two promoters are distinct promoter sequences. In some aspects, the vector comprises a first AAV inverted terminal repeat (ITR) sequence, a first promoter sequence, a first nucleic acid element, a second RNA promoter sequence, a second nucleic acid element, and a second ITR sequence, wherein the first promoter sequence and second promoter sequence are distinct promoter sequences.

Vectors of the disclosure can further comprise one or more additional nucleic acid elements.

In some aspects, vectors of the disclosure comprise a total of at least one, at least two, at least three, at least four, at least five, at least six, at least seven, at least eight, at least nine, or at least ten nucleic acid elements. In some aspects, vectors of the disclosure comprise one, two, three, four, five, six, seven, eight, nine, or ten nucleic acid elements.

In some aspects, vectors of the disclosure comprise a total of at least one, at least two, at least three, at least four, at least five, at least six, at least seven, at least eight, at least nine, or at least ten noncoding RNA molecules. In some aspects, vectors of the disclosure comprise one, two, three, four, five, six, seven, eight, nine, or ten noncoding RNA molecules.

In some aspects, vectors of the disclosure comprise a total of at least one, at least two, at least three, at least four, at least five, at least six, at least seven, at least eight, at least nine, or at least ten snRNA molecules. In some aspects, vectors of the disclosure comprise one, two, three, four, five, six, seven, eight, nine, or ten snRNA molecules.

In some aspects, vectors of the disclosure comprise a total of at least one, at least two, at least three, at least four, at least five, at least six, at least seven, at least eight, at least nine, or at least ten sgRNA molecules. In some aspects, vectors of the disclosure comprise one, two, three, four, five, six, seven, eight, nine, or ten sgRNA molecules.

In some aspects, vectors of the disclosure comprise a total of at least one, at least two, at least three, at least four, at least five, at least six, at least seven, at least eight, at least nine, or at least ten protein encoding transgenes. In some aspects, vectors of the disclosure comprise one, two, three, four, five, six, seven, eight, nine, or ten protein encoding transgenes.

In some aspects, each nucleic acid element, such as a noncoding RNA, snRNA molecule, sgRNA molecule, or transgene is operably linked to a promoter sequence. In some aspects, a single promoter controls expression of two or more nucleic acid elements. In some aspects, when a single promoter is operably linked to two or more nucleic acid elements, such as a noncoding RNA molecule or transgene, a spacer sequence separates the elements.

In some aspects, vectors of the disclosure comprise one, two, three, four, five, six, seven, eight, nine, or ten promoter sequences.

In some aspects, vectors of the disclosure comprising two or more promoter sequences comprise promoters distinct from the other promoters in the vector.

In some aspects, each promoter sequence in a vector lacks significant sequence identity to other sequences of the vector including other promoter sequences of the vector. In some aspects, a promoter sequence of the vector has less than about 75%, about 70%, about 65%, about 60%, about 55%, about 50%, about 45%, about 40%, about 35%, about 30%, about 25%, about 20%, about 15%, about 10%, about 5%, about 1%, or about 0% sequence identity to other sequences of the vector including other promoter sequences. In some aspects, a promoter sequence of the vector has less than about 75% sequence identity to other sequences of the vector including other promoter sequences. In some aspects, a promoter sequence of the vector has less than about 50% sequence identity to other sequences of the vector including other promoter sequences. In some aspects, a promoter sequence of the vector has less than about 45% sequence identity to other sequences of the vector including other promoter sequences. In some aspects, a promoter sequence of the vector has less than about 40% sequence identity to other sequences of the vector including other promoter sequences. In some aspects, a promoter sequence of the vector has less than about 35% sequence identity to other sequences of the vector including other promoter sequences. In some aspects, a promoter sequence of the vector has less than about 30% sequence identity to other sequences of the vector including other promoter sequences.

The term “homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than about 40%-70% identity, or alternatively less than about 60%, about 50%, about 30%, or about 25% identity, with another sequence.

Without wishing to be bound by theory, vectors, including AAV vectors and rAAV vectors of the disclosure, having repetitive sequences (including promoter sequences) are capable of forming complementary interactions between said repeated sequences. Such complementary interactions include base-pairing and hybridization interactions. Said interactions form loops and intermolecular and intramolecular interactions. Said self-complementary interactions result in poor packaging efficiency and truncations of the final packaged vector (such as a packaged rAAV vector in an AAV viral vector).

As such, eliminating sequences with high sequence identity and/or similarity within a single vector will eliminate these self-complementary interactions leading to enhanced packaging efficiency. This enhanced packaging efficiency produces purer viral particles with fewer undesired vector species (i.e. truncated vector species). As such, the resulting viral particles, such as AAV viral particles, can lead to enhanced expression and efficacy in a therapeutic context in in vivo, ex vivo, and clinical settings.

Further, vectors, including rAAV vectors, described herein yield more reproducible viral packaging and manufacturing. Reducing potential for truncated genome species allows for a more accurate determination of viral titer to expression relationship. The amount of truncation events may vary from one viral production run to another, so the relationship between titer and expression (potency/safety) must be determined for each production run. Eliminating truncation events will reduce the variation between production batches.

Because vectors of the disclosure comprise fewer repetitive elements that may allow for self-commentary interactions within the vector, vectors of the disclosure exhibit greater expression than vectors that do comprise self-complementary interactions, for instance due to the use of repeated promoter sequences. Further, vectors of the disclosure that comprise fewer repetitive elements are much less likely to exhibit truncations and/or deletions within the vector sequence. Accordingly, vectors such as rAAV vectors of the disclosure packaged as AAV viral vectors will have higher efficiency production of the correct rAAV vector (i.e. will not have truncations or deletions of the desired rAAV vector comprising nucleic acid elements of the disclosure such as snRNA or transgenes).

In some aspects, the AAV viral vector exhibits greater expression in a subject or cell relative to an AAV viral vector comprising an rAAV vector comprising a single noncoding RNA or transgene or an rAAV vector comprising repeated promoter sequences operably linked to noncoding RNA molecules or transgene sequences.

The term “hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

### Nucleic Acid Elements

The disclosure provides vectors, including rAAV vectors, comprising one or more nucleic acid elements operably linked to promoter sequences. Nucleic acid elements can be any nucleic acid sequence including transgene sequences encoding for proteins and/or peptides. Proteins of the disclosure can be any protein known in the art. In some aspects, the protein is a therapeutic protein. In some aspects, the protein is a synthetic or non-naturally occurring protein.

In some aspects, the nucleic acid element is a noncoding RNA. In some aspects, the noncoding RNA is an RNA binding noncoding RNA. In some aspects, the noncoding RNA is a small-nuclear RNA (snRNA) molecule, a single guide RNA molecule (sgRNA), a microRNA, a short hairpin RNA (shRNA), an enhancer RNA (eRNA), a small nucleolar RNA (snoRNA), or a long noncoding RNA (lncRNA). In some aspects, the sgRNA is used in conjunction with CRISPR/Cas systems to target, bind, and/or cleave nucleic acids including DNA and RNA sequences. In some aspects, the noncoding RNA is an snRNA molecule. Short nuclear RNA molecules of the disclosure can be non-natural, modified, and/or engineered snRNA molecules. In some aspects, the snRNA molecules of the disclosure bind and target RNA molecules.

In some aspects, the length of each of the first nucleic acid elements, the second nucleic acid element, and the one or more additional nucleic acid elements is between about 10 and 5,000 nucleotides. In some aspects, the length of each of the first nucleic acid elements, the second nucleic acid element, and the one or more additional nucleic acid elements is between about 50 and 5,000 nucleotides. In some aspects, the length of each of the first nucleic acid elements, the second nucleic acid element, and the one or more additional nucleic acid elements is between about 10 and 2,500 nucleotides. In some aspects, the length of each of the first nucleic acid elements, the second nucleic acid element, and the one or more additional nucleic acid elements is between about 50 and 2,500 nucleotides. In some aspects, the length of each of the first nucleic acid elements, the second nucleic acid element, and the one or more additional nucleic acid elements is between about 10 and 1,000 nucleotides. In some aspects, the length of each of the first nucleic acid elements, the second nucleic acid element, and the one or more additional nucleic acid elements is between about 10 and 750 nucleotides. In some aspects, the length of each of the first nucleic acid elements, the second nucleic acid element, and the one or more additional nucleic acid elements is between about 10 and 500 nucleotides. In some aspects, the length of each of the first nucleic acid elements, the second nucleic acid element, and the one or more additional nucleic acid elements is between about 10 and 250 nucleotides.

In some aspects, there are from about 10 to about 5,000 nucleotides between the 3′ end of the first promoter and the 5′ start of the second promoter. In some aspects, there are from about 50 to about 5,000 nucleotides between the 3′ end of the first promoter and the 5′ start of the second promoter. In some aspects, there are from about 10 to about 2,500 nucleotides between the 3′ end of the first promoter and the 5′ start of the second promoter. In some aspects, there are from about 10 to about 1,000 nucleotides between the 3′ end of the first promoter and the 5′ start of the second promoter. In some aspects, there are from about 10 to about 500 nucleotides between the 3′ end of the first promoter and the 5′ start of the second promoter. In some aspects, there are from about 10 to about 250 nucleotides between the 3′ end of the first promoter and the 5′ start of the second promoter.

snRNA Molecules

The disclosure provides improved rAAV vectors for packaging snRNA molecules.

Small nuclear RNA (snRNA) is one of the smallest types of RNA with an average size of about 150 nucleotides. snRNAs are functional non-coding RNAs. Eucaryotic genomes code for a variety of non-coding RNA such as snRNA, a class of highly abundant RNA, localized in the nucleus with important functions in intron splicing and RNA processing. snRNA, in the pre-mRNA splicing process, are capable of forming ribonucleoprotein particles (snRNPs) along with other proteins. These snRNPs and additional proteins form a large particulate complex (spliceosome) bound to the unspliced pre-mRNA transcripts. In addition to splicing, snRNAs function in nuclear maturation of nascent transcripts, gene expression regulation, as a splice donor in non-canonical systems, and in 3′ end processing of replication-dependent histone mRNAs. U7 snRNA can be programmed to bind and modulate mRNA without exogenous protein expression but there still exists a need to develop a highly specific mRNA-targeting therapeutic that minimizes immunogenic risk. Furthermore, the small size of these programmed snRNAs creates an opportunity to develop single vector, highly specific (allele-specific), single target and multi-targeting gene therapy approaches.

In some aspects, the disclosure provides gene therapy compositions comprising engineered snRNA (esnRNA) comprising an engineered snRNA stem loop (eSL).

Small nuclear ribonucleic acids (snRNAs) are essential components of small nuclear ribonucleoprotein complexes (snRNPs) which, when assembled with additional proteins, form the large ribonucleoprotein complex known as the spliceosome, the cell machinery appointed to mediate the entire mRNA maturation process. The spliceosome is responsible for precursor mRNA splicing; the process that removes introns from RNA transcripts before protein production. An individual snRNA is generally about 250 nucleotides or less in size. For example, U1 snRNA is 164 nucleotides in length and is encoded by genes that occur in several copies within the human genome. U1 snRNA represents the ribonucleic component of the nuclear particle U1 snRNP. The U1 snRNA has a stem and loop tridimensional structure and within the 5′ region there is a single-stranded sequence, generally about 9 nucleotides in length, capable of binding by complementary base pairing to the splicing donor site on the pre-mRNA molecule. (Horowitz et al., 1994, Trends Genet., 10(3):100-6.) The various spliceosomal snRNAs have been designated as U1, U2, U4, U5, U6, U4ATAC, U6ATAC, U7, U11 and U12, due to the generous amount of uridylic acid they contain. (Mattaj et al., 1993, FASEB J, 15, 7:47-53.)

snRNA systems can be used for treating toxic mutations. For example, antisense oligonucleotides that interfere with splice sites and regulatory elements within an exon containing toxic mutations, induce skipping of specific exons at the pre-RNA level.

Such antisense sequences can be delivered using viral vectors carrying a gene from which the antisense sequence via an snRNA can be transcribed. U7 snRNA is endogenously involved in histone pre-mRNA 3′-end processing, but can be converted into a versatile tool for splicing modulation by a small change in the binding site for Sm/Lsm proteins. One such therapeutic strategy for treating DMD (Duchenne muscular dystrophy) has used modified U7 snRNA to convert an out-of-frame mutation into an in-frame mutation, which gives rise to internally deleted toxic RNA, but still functional dystrophin. (Goyenvalle et al., 2009, 17(7): 1234-1240.)

Most U-rich snRNPs are complexes that mediate the splicing of pre-mRNAs. U7 snRNP is an exception. U7 is not involved in splicing but rather is a key factor in the unique 3′-end processing of replication-dependent histone mRNAs. By modifying the U7 snRNA histone binding sequence and the Sm motif, U7 can no longer be involved in processing the histone pre-mRNA and instead targets pre-mRNAs or smRNA for blocking or splicing modulation. In this manner, U7 snRNA can be used as an effective gene therapy platform. A U7 snRNA platform also has the additional advantages of being a compact size, having the capability to accumulate in the nucleus without causing cellular toxicity, and possesses little to no immunoreactivity. (Gadgil et al., 2021, J Gene Med, 23(4): e3321.)

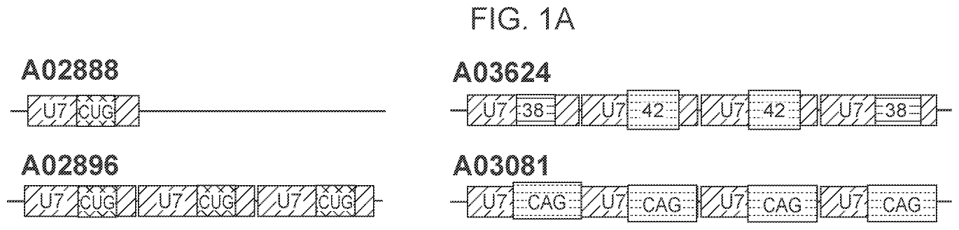

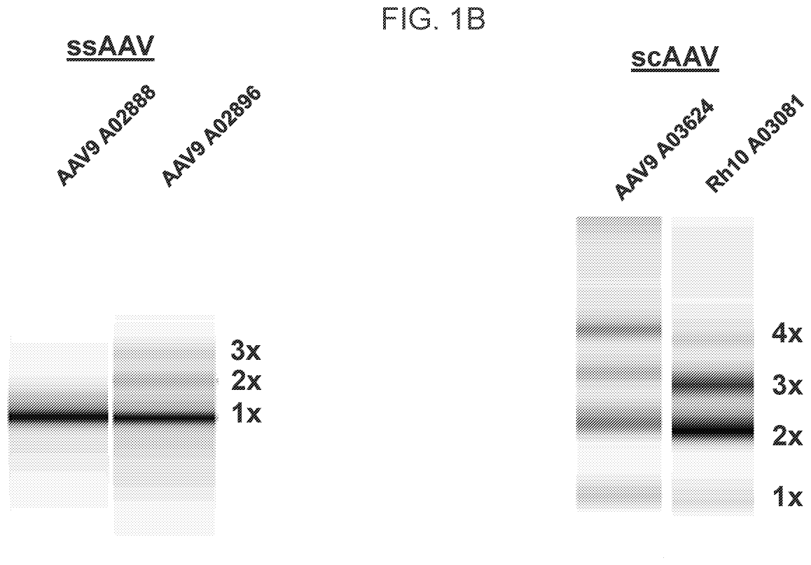

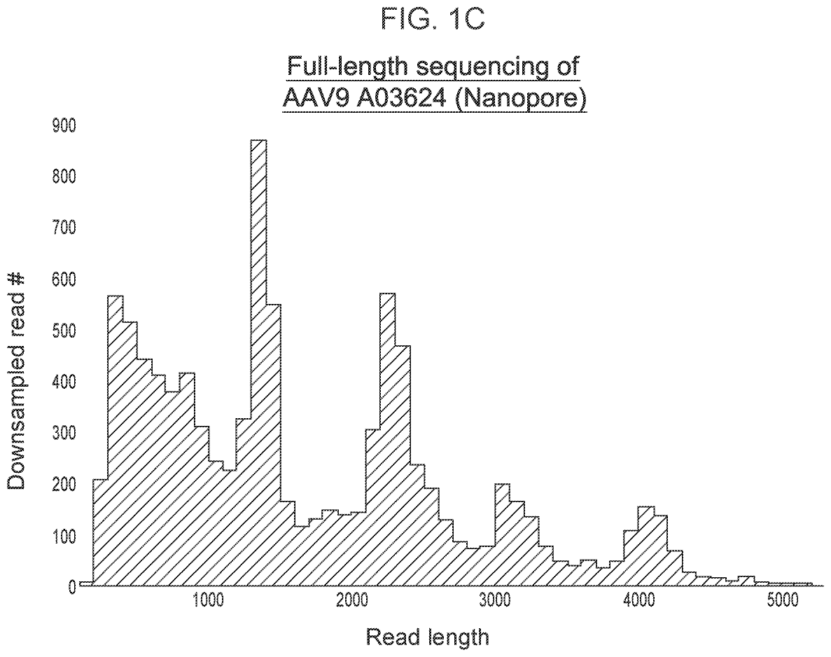

Disclosed herein is a newly engineered and redesigned snRNA platform (or esnRNA platform) comprising an 1) engineered stem loop (eSL). Compensatory modifications made to the native stem loop sequence create an engineered stem loop (eSL) which more effectively communicates (folds and anneals) with the snRNA interaction stabilization domain (ISD) which in turn creates a snRNA platform with increased stability. See FIG. 1. U7 snRNAs have been previously shown to be programmable to modulate mRNAs. Disclosed herein are programmed engineered snRNA improvements which are capable of being used as a gene therapy tool. These engineered snRNA systems are shown herein to lead to blocking microsatellite repeat expansions (shown herein for treating myotonic dystrophy (DM1) or Huntington's disease (HD)) and splicing modulation (shown herein for treating USH2A (Usher Syndrome type 2). In one embodiment, these snRNA are human snRNAs. In one embodiment the U7 is a human U7. In another embodiment disclosed herein, engineered snRNA comprises varying types of snRNAs (U1-U12, etc.) by combining domains of endogenous snRNAs to fine tune stabilization of the platform and/or to reduce off-target effects. For example, in one embodiment, the engineered snRNA system comprises a combination of human U7 and human U1 snRNA components.

Additional elements that can tune the processing and abundance of the RNA can be further engineered into the esnRNAs comprising eSLs. See FIG. 1C. In one embodiment, additional elements that can tune the processing, stability, and abundance of the esnRNA can be further engineered into the esnRNAs at the 5′ or 3′ ends. In another embodiment, such elements may include but are not limited to stem loops, hairpins, G-C clamps, kissing loops, triplexes, quadruplexes, and protein binding sites.

snRNA of the disclosure can be programmed to comprise a targeting sequence (TS) that targets one or more RNAs of interest. In one example, U7 snRNA can be programmed by replacing the histone mRNA binding sequence with a sequence complementary to a target of interest. The exemplary esnRNA shown herein lead to blocking microsatellite repeat expansions (for treating myotonic dystrophy (DM1) or Huntington's disease (HD)) and splicing modulation (for treating USH2A (Usher Syndrome type 2)). In one embodiment, the target RNA of interest is a microsatellite repeat RNA or a non-repeat RNA. In another embodiment, the microsatellite repeat RNA of interest is selected from the group consisting of CUG, CAG, GGGGCC, and CCCCGG. In one embodiment, the esnRNA comprises a targeting sequence that targets two target RNAs of interest are GGGGCC and CCCCGG. In another embodiment, the two target RNAs of interest are a microsatellite repeat RNA and a non-repeat RNA. In another embodiment, the non-repeat RNA is a flanking sequence to the microsatellite repeat RNA. In another embodiment, the esnRNA comprises a targeting sequence that targets two or more RNAs of interest. In another embodiment the esnRNA comprises two or more targeting sequences (TS) that target two or more RNAs of interest. In an embodiment, the targeting sequence(s) (TS) can be located in a 5′ (5′TS) position in the snRNA construct. In an alternative embodiment, the targeting sequence(s) (TS) can be located in a 3′ (3′TS) position in the snRNA construct, particularly if the snRNA construct is not a U7-based snRNA.

### Engineered Stem Loops

snRNA and engineered snRNA (esnRNA) systems disclosed herein can comprise an engineered stem loop (eSL) which includes compensatory modifications to a native snRNA stem loop. These modifications result in increased stability of the esnRNP compared to snRNP comprising an unmodified stem loop. An eSL disclosed herein can be derived from any snRNP U1-U12. In one embodiment, the eSL is a U7 eSL. In one embodiment, the eSL is a human or mouse U7 eSL. In one embodiment, the eSL is a human eSL. In one embodiment, the eSL is a mouse eSL. In some embodiments, the eSL is a human and mouse eSL. In some embodiments, the eSL is a non-human eSL, e.g., a mouse eSL, a pig, a sheep eSL, a goat eSL, a cow eSL, a dog eSL, a cat eSL, a horse eSL, or a combination thereof. In some embodiments, the eSL sequence is not a native stem loop sequence. In some embodiments, the nucleic acid sequence of the eSL is at least 65%, 70%, 75%, 80%, 85%, 90%, 95%, 96%, 97%, 98%, 99% or 100% (or any percentage in between) is not a native stem loop sequence. Engineered stem loops are described in WO2023168458, the contents of which are incorporated herein by reference in its entirety for examples of eSL sequences that may be used in the constructs described herein.

In another embodiment, the eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 57)
ggctttctggctccttaccggaaagcc,
(SEQ ID NO: 58)
ggctttctgggaggttaccggaaagcc,
(SEQ ID NO: 59)
ggctttctggcctccttaccggaaagcc,
(SEQ ID NO: 60)
ggctttctggggaggttaccggaaagcc,
(SEQ ID NO: 61)
ggctttctggctggctaccggaaagcc,
(SEQ ID NO: 62)
ggctttctggcttccccggaaagcc,
(SEQ ID NO: 63)
ggctttctggcttcttcccggaaagcc,
(SEQ ID NO: 64)
ggctttctggcaacttaccggaaagcc,
(SEQ ID NO: 65)
ggctttctggttcggtaccggaaagcc,
(SEQ ID NO: 66)
ggctttctggaagccttaccggaaagcc,
(SEQ ID NO: 67)
ggctttctggcttcttaccggaaagcc,
or
(SEQ ID NO: 68)
GGCTTTCTGGCCTCCGCCGGAAAGCCCCT.

In some embodiments, a human eSL comprises the sequence set forth in SEQ ID NO: 57. In some embodiments, a human eSL comprises the sequence set forth in SEQ ID NO: 58. In some embodiments, a human eSL comprises the sequence set forth in SEQ ID NO: 59. In some embodiments, a human eSL comprises the sequence set forth in SEQ ID NO: 60. In some embodiments, a human eSL comprises the sequence set forth in SEQ ID NO: 61. In some embodiments, a human eSL comprises the sequence set forth in SEQ ID NO: 62. In some embodiments, a human eSL comprises the sequence set forth in SEQ ID NO: 63. In some embodiments, a human eSL comprises the sequence set forth in SEQ ID NO: 64. In some embodiments, a human eSL comprises the sequence set forth in SEQ ID NO: 65. In some embodiments, a human eSL comprises the sequence set forth in SEQ ID NO: 66. In some embodiments, a human eSL comprises the sequence set forth in SEQ ID NO: 67. In some embodiments, a human eSL comprises the sequence set forth in SEQ ID NO: 68.

In some embodiments, a murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 69)
ggctttctggctccttaccggaaagcccct
(SEQ ID NO: 70)
Ggttttctgacctccgtcggaaaacccct,
(SEQ ID NO: 71)
ggttttctgacctccttcggtcggaaaacccct,
(SEQ ID NO: 72)
Ggttttctgacctccgtcggaaaacc,
(SEQ ID NO: 73)
GGTTTTCTGACACTCCGTCGGAAAACCCCT,
(SEQ ID NO: 74)
GGTTTTCTGATCTCCATCGGAAAACCCCT,
or
(SEQ ID NO: 75)
GGTTTTCCGACCTCCGTCGGAAAACCCCT.

In some embodiments, a murine eSL comprises the sequence set forth in SEQ ID NO: 69. In some embodiments, a murine eSL comprises the sequence set forth in SEQ ID NO: 70. In some embodiments, a murine eSL comprises the sequence set forth in SEQ ID NO: 71. In some embodiments, a murine eSL comprises the sequence set forth in SEQ ID NO: 72. In some embodiments, a murine eSL comprises the sequence set forth in SEQ ID NO: 73. In some embodiments, a murine eSL comprises the sequence set forth in SEQ ID NO: 74. In some embodiments, a murine eSL comprises the sequence set forth in SEQ ID NO: 75.

In some embodiments, a human or murine eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 76)
GGCTTTCTGGCACTCCACCGGAAAGCCCCT,
(SEQ ID NO: 77)
GGCTTTCTGGCACTCCGCCGGAAAGCCCCT,
or
(SEQ ID NO: 78)
GGCTTTCTGGCCTCCACCGGAAAGCCCCT.

In some embodiments, a human or murine eSL comprises the sequence set forth in SEQ IID NO: 76. In some embodiments, a human or murine eSL comprises the sequence set forth in SEQ IID NO: 10477 In some embodiments, a human or murine eSL comprises the sequence set forth in SEQ IID NO: 78.

In some embodiments, a dog or cat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to the nucleotide sequence

(SEQ ID NO: 79)
GGTTTTCCGGTCTCCACCGGAAAGCCCCC.

In some embodiments, a cow, sheep, or goat eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 80)
GGCTTTCCGGTCTCCACCGGAAAGCCCCT,
or
(SEQ ID NO: 81)
GGCTTTCCGGCCTCCGCCGGAAAGCCCCT.

In some embodiments, a cow, sheep, or goat eSL comprises the sequence set forth in SEQ ID NO: 80. In some embodiments, a cow, sheep, or goat eSL comprises the sequence set forth in SEQ ID NO: 81.

In some embodiments, a pig eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 82)
GGTTTTCCGGTCTCCACCGGAAAACCCTT,
(SEQ ID NO: 83)
GGTTTTCCGTGCTCCCACGGAAAACCCTT,
(SEQ ID NO: 84)
GGTTTTCCGGCCTCCGCCGGAAAACCCTT,
(SEQ ID NO: 85)
GGTTTTCCGTGACTCCCACGGAAAACCCTT,
or
(SEQ ID NO: 86)
GGTTTTCCGGCACTCCGCCGGAAAACCCTT.

In some embodiments, a pig eSL comprises the sequence set forth in SEQ ID NO: 82. In some embodiments, a pig eSL comprises the sequence set forth in SEQ ID NO: 83. In some embodiments, a pig eSL comprises the sequence set forth in SEQ ID NO: 84. In some embodiments, a pig eSL comprises the sequence set forth in SEQ ID NO: 85. In some embodiments, a pig eSL comprises the sequence set forth in SEQ ID NO: 86.

In some embodiments, a horse eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 87)
GGTCTTCCGGTCTCCTCCGGAAGGCCCCC,
or
(SEQ ID NO: 88)
GGTCTTCCGGCTCCCCGGAAGGCCCCC.

In some embodiments, a horse eSL comprises the sequence set forth in SEQ ID NO: 87. In some embodiments, a horse eSL comprises the sequence set forth in SEQ ID NO: 88.

In some embodiments, a sheep eSL comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 89)
GGCTTTCCGTGCTCCCACGGAAAGCCCCT,
(SEQ ID NO: 90)
GGCTTTCCGTGACTCCCACGGAAAGCCCCT,
or
(SEQ ID NO: 91)
GGCTTTCCGGCACTCCGCCGGAAAGCCCCT

In some embodiments, a sheep eSL comprises the sequence set forth in SEQ ID NO: 89. In some embodiments, a sheep eSL comprises the sequence set forth in SEQ ID NO: 90. In some embodiments, a sheep eSL comprises the sequence set forth in SEQ ID NO: 91.

In some embodiments, engineered stem loops provide for enhanced stability of an snRNA relative to an snRNA comprising a native stem loop. In some embodiments is a native snRNA stem loop comprises, consists essentially of, or consists of a nucleic acid sequence at least 65%, 70%, 75%, 80%, 85%, 90%, 95%, 96%, 97%, 98%, 99% or 100% (or any percentage in between) identical to one or more of the following nucleotide sequences:

(SEQ ID NO: 92)
Ggttttctgacttcggtcggaaaacccct,
(SEQ ID NO: 93)
ggttttctgacttcggtcggaaaacc,
(SEQ ID NO: 94)
Ggctttctggctttttaccggaaagcc,
(SEQ ID NO: 95)
ggctttctggctttttaccggaaagccCCT,
(SEQ ID NO: 96)
GGCTTTCCGGCCTCCGCCGGAAAGCCCCT,
or
(SEQ ID NO: 97)
GGCTTTCCGGCCTCCGCCGGAAAGCC.

In some embodiments is a native snRNA stem loop comprises the sequence set forth in SEQ ID NO: 92. In some embodiments is a native snRNA stem loop comprises the sequence set forth in SEQ ID NO: 93. In some embodiments is a native snRNA stem loop comprises the sequence set forth in SEQ ID NO: 94. In some embodiments is a native snRNA stem loop comprises the sequence set forth in SEQ ID NO: 95. In some embodiments is a native snRNA stem loop comprises the sequence set forth in SEQ ID NO: 96.

### 5′ Interaction Stability Domain

The eSL disclosed herein possesses more effective folding and annealing properties with a 5′ interaction stability domain (5′ISD) and this in turn results in increased stability of the esnRNA compared to a non-engineered snRNA. The 5′ ISD has nucleotides that are complementary to the nucleotides within the engineered SL, and without wishing to be bound by theory, an interaction between the 5′ISD and eSL is predicted to form secondary structure that protects the 5′ end of an snRNA. In some aspects the 5′ ISD anneals and/or hybridizes to an eSL of the disclosure. In some aspects the 5′ISD is a sequence having complementarity and/or reverse complementarity to a sequence present in an eSL of the disclosure. In some aspects a 5′ISD disclosed herein can comprise or consist of one of the following nucleotide sequences:

ggagt,
cctct,
ggaggt,
cctcct,
agccag,
ggaag,
gaagaag,
gttg,
ccgaa,
taaggag,
gaag,
or
ggctt.

### Sm Binding Domains

The snRNA systems disclosed herein utilize an Sm binding domain (SmBD). The Sm protein ring that assembles around the Sm binding domain (SmBD) to form an snRNP includes SmB/B′, SmD1, SmD2, SmD3, SmE, SmF, and SmG. The U7 Sm binding site recruits endogenous RNA binding factors and can be replaced with a non-U7 SmBD to make the esnRNA more stable. In one embodiment, the SmBD is selected from the group consisting of U1, U2, U4, and U5 snRNAs. In another embodiment, the SmBD is derived from a pseudo snRNA. In another embodiment, the SmBD is a nucleotide sequence comprising SEQ ID NO: 78 (ATTTTT). In another embodiment, the SmBD comprises a nucleotide sequence selected from the group consisting of AATTTTTGG, AATTTGTGG, AATTTGTGG, AATTTCTGG, GATTTTTGG, AATTTTTGA, AATTTTTTG, AATTTTTGGAGCA (SEQ ID NO: 105), or AATTTTTGGAGTA (SEQ ID NO: 106).

### Promoter Sequences

A “promoter” is a control sequence that is a region of a polynucleotide sequence at which initiation and rate of transcription are controlled. It may contain genetic elements at which regulatory proteins and molecules may bind such as RNA polymerase and other transcription factors.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, with the incorporation of coding sequences into the compositions disclosed herein, multicistronic vectors can simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or other sequences encoding the self-cleaving peptides.

### Noncoding RNA Promoter Sequences

The noncoding RNA promoters of rAAV vectors disclosed herein can comprise an snRNA promoter from any of U1-U12. In some embodiments, the U1-U12 promoters are derived from any species including human and mouse. In one embodiment, the snRNA promoter is a U7 promoter. In another embodiment, the U7 promoter is a human U7 promoter (hU7) or a mouse U7 promoter (mU7). In another embodiment, the U1 promoter is a human U1 promoter (hU1) or a mouse U1 promoter (mU1). In another embodiment, the U4 promoter is a human U4 promoter (hU4) or a mouse U4 promoter (mU4). In another embodiment, the U5 promoter is a human U5 promoter (hU5) or a mouse U5 promoter (mU5).

In some aspects, a noncoding RNA promoter is a snoRNA promoter. In one embodiment, the snoRNA promoter is a human snoRNA promoter. In another embodiment, the snoRNA promoter is a U3 snoRNA promoter.

In other aspects, the snRNA promoter comprises, consists essentially of, or consists of a nucleic acid sequence at least 65%, 70%, 75%, 80%, 85%, 90%, 95%, 96%, 97%, 98%, 99% or 100% (or any percentage in between) identical to a promoter and/or promoter sequence listed in the Exemplary Promoter Table which follows:

| Exemplary | |
|---|---|
| Promoter | Promoter Sequence |
| hU1 | AAGGACCAGCTTCTTTGGGAGAGAACAGACGCAGGGGCGGGAGGGA |
| | AAAAGGGAGAGGCAGACGTCACTTCCCCTTGGCGGCTCTGGCAGCAG |
| | ATTGGTCGGTTGAGTGGCAGAAAGGCAGACGGGGACTGGGCAAGGC |
| | ACTGTCGGTGACATCACGGACAGGGCGACTTCTATGTAGATGAGGCA |
| | GCGCAGAGGCTGACGTCTTCGCCACTTGCTGCTTCACCACGAAGGAG |
| | TTCCCGTGCCCTGGGAGCGGGTTCAGGACCGCTGATCGGAAGTGAGA |
| | ATCCCAGCTGTGTGTCAGGGCTGGAAAGGGCTCGGGAGTGCGCGGGG |
| | CAAGTGACCGTGTGTGTAAAGAGTGAGGCGTATGAGGCTGTGTCGGG |
| | GCAGAGGCacaacgtttc |
| | (SEQ ID NO: 3) |
| | TAAGGACCAGCTTCTTTGGGAGAGAACAGACGCAGGGGGGGGAGGG |
| | AAAAAGGGAGAGGCAGACGTCACTTCCCCTTGGCGGCTCTGGCAGCA |
| | GATTGGTCGGTTGAGTGGCAGAAAGGCAGACGGGGACTGGGCAAGG |
| | CACTGTCGGTGACATCACGGACAGGGCGACTTCTATGTAGATGAGGC |
| | AGCGCAGAGGCTGACGTCTTCGCCACTTGCTGCTTCACCACGAAGGA |
| | GTTCCCGTGCCCTGGGAGCGGGTTCAGGACCGCTGATCGGAAGTGAG |
| | AATCCCAGCTGTGTGTCAGGGCTGGAAAGGGCTCGGGAGTGCGCGGG |
| | GCAAGTGACCGTGTGTGTAAAGAGTGAGGCGTATGAGGCTGTGTCGG |
| | GGCAGAGGCacaacgtttc (SEQ ID NO: 4) |
| hU2 | CCACGCCCTCTGTGAAAGGGCGGGGCATGCAAATTCGAAATGAAAGC |
| | CCGGGAACGCCGGAAGAAGCACGGGTGTAAGATTTCCCTTTTCAAAG |
| | GCAGAGAATAAGAAATCAGCCCGAGAGTGTAAGGGCGTCAATAGCG |
| | CTGTGGACGAGACAGAGGGAATGGGGCAAGGAGCGAGGCTGGGGCT |
| | CTCACCGCGACTTGAATGTGGATGAGAGTGGGACGGTGACGGCGGGC |
| | GCGAAGGCGAGCGC |
| | SEQ ID NO: 5) |
| hU4 | TTCGCAGTCTCTGAATTAAGTCTATTAGCATGTTCCTCCCATAGTGCT |
| | TTGCTTCATATCAACAAAAACCTAGCTAAGTGAAATCAGCAACGATA |
| | TGCAGAAACCACCTACGCAGGTCACAAACATCTTTCTATGATTGTATA |
| | ATTTTCAAGCAAGCAATAAGTGAAGATTTTTCCATAGGCCCTAAACTC |
| | ACCTTTGCGAAATAGGAAGCTGGTTTATTGGGAGTGATGAGCAGGGG |
| | GCGTAACAAATT |
| | (SEQ ID NO: 6) |
| hU5 | GCAGCAAGGCCTCCACTTCACCCCCTAAAGGTTGCCCCAAGAGCACC |
| | GTGTGACTGCTAAGGTATTTCCGGAGTCTAAAGACGATTATTCAGGTC |
| | TCATTTGCATACCCATAATACACTGCAAACAGTATTTTTTTCGGAAAA |
| | ACATTTATATATTGCTTGACATTTTTAAGTATGAGAATTTTGCATGCA |
| | GAATTTTTTTGTATAAACTTTCTCAGGTAGTAACCCTTGGGATTAGTA |
| | GACACCATCAGTGTACTAGGAATTGCAGTTACCCGAAAATTGAGTTA |
| | CAGAAGTAACTGGT |
| | SEQ ID NO: 7) |
| bU6 | Gtttattacagggacagcagagatccagtttggttaattaaggtaccgagggcctatttcccatgattccttcatatttg |
| | catatacgatacaaggctgttagagagataattagaattaatttgactgtaaacacaaagatattagtacaaaatacgt |
| | gacgtagaaagtaataatttcttgggtagtttgcagttttaaaattatgttttaaaatggactatcatatgcttaccgtaact |
| | tgaaagtatttcgatttcttggctttatatatcttgtggaaaggacgaaacacc |
| | (SEQ ID NO: 8) |
| h7sk | CTGCAGTATTTAGCATGCCCCACCCATCTGCAAGGCATTCTGGATAGT |
| | GTCAAAACAGCCGGAAATCAAGTCCGTTTATCTCAAACTTTAGCATTT |
| | TGGGAATAAATGATATTTGCTATGCTGGTTAAATTAGATTTTAGTTAA |
| | ATTTCCTGCTGAAGCTCTAGTACGATAAGTAACTTGACCTAAGTGTAA |
| | AGTTGAGATTTCCTTCAGGTTTATATAGCTTGTGCGCCGCCTGGGTAC |
| | CTC |
| | (SEQ ID NO: 9) |
| tRNA(Val) | CAGGACTAGTCTTTTAGGTCAAAAAGAAGAAGCTTTGTAACCGTTGG |
| | TTTCCGTAGTGTAGTGGTTATCACGTTCGCCTAACACGCGAAAGGTCC |
| | CCGGTTCGAAG |
| | (SEQ ID NO: 10) |
| mU1 | TTGTTCCTCTTAGTGTTAATTCACACTAAAGACTGTGCATCCGACTCC |
| | TACATTTATGAAAGTAAATGCCTGTTGTTAGAACAAAAAAGGCTACA |
| | GAACAAAAAACAAAGCGAAATACCATCTGCTTTAGGTTCAGTGTGGT |
| | ATTTTCCCGCTGACAGGGAGGCGGGTTTTTGGGTACAGGAAACGAGT |
| | CACTATGGAGGCGGTACTATGTAGATGAGAATTCAGGTGCAAACTGG |
| | GAAAAGCAACTGCTTCCAAATATTTGTGATTTTTACAGTGTAGTTTTG |
| | GAAAAACTCTTAGCCTACCAATTCTTCTAAGTGTTTTAAAATGTGGGA |
| | GCCAGTACACATGAAGTTATAGAGTGTTTTAATGAGGCTTAAATATTT |
| | ACCGTAACTATGAAATGCTACGCATATCATGCTGTTCAGGCTCCGTGG |
| | CCACGCAACTC |
| | SEQ ID NO: 11) |
| mU7 | TaacaacataggagctgtgattggctgttttcagccaatcagcactgActcatttgcatagcctttacaagcggtcac |
| | aaactcaagaaacgagcggttttaatagtcttttagaatattgtttatcgaaccgaataaggaactgtgctttgtgattca |
| | catatcagtggaggggtgtggaaatggcaccttgatctcaccctcatcgaaagtggagttgatgtcctTccctggct |
| | cgctacagacgcacttccgcaa |
| | (SEQ ID NO: 12) |
| mU7 + | TaacaacataggagctgtgattggctgttttcagccaatcagcactgActcatttgcatagcctttacaagcggtcac |
| extra 3′ | aaactcaagaaacgagcggttttaatagtcttttagaatattgtttatcgaaccgaataaggaactgtgctttgtgattca |
| | catatcagtggaggggtgtggaaatggcaccttgatctcaccctcatcgaaagtggagttgatgtcctTccctggct |
| | cgctacagacgcacttccgcaa (SEQ ID NO: 13) |
| bU7 | TACTGCCGAATCCAGGTCTCCGGGCTTAACAACAACGAAGGGGCTGT |
| | GACTGGCTGCTTTCTCAACCAATCAGCACCGAACTCATTTGCATGGGC |
| | TGAGAACAAATGTTCGCGAACTCTAGAAATGAATGACTTAAGTAAGT |
| | TCCTTAGAATATTATTTTTCCTACTGAAAGTTACCACATGCGTCGTTG |
| | TTTATACAGTAATAGGAACAAGAAAAAAGTCACCTAAGCTCACCCTC |
| | ATCAATTGTGGAGTTCCTTTATATCCCATCTTCTCTCCAAACACATAC |
| | GCA |
| | (SEQ ID NO: 1) |
| mU5 | TGCCAACAGCTTTGAAATCCTCTGTGCTCTTGTGCCAATCCCACCCTC |
| | AGAACAGGGAGTCTGTCGGTTATGTTTTCGTCCTTTACAGTTCTTCGC |
| | CGTCTAATCCATTGGAAACCTCCTTTATTTAAAGGAAACCGTGTCCAA |
| | ATTCAGTGTACAGAGGTGCAAACCTCCGCCCCGCTCCTTGTAGGAAG |
| | CCCACGGGAACTGGGCCAATCGGACGGCGCTTACTCGGCTCATTTAC |
| | ATACCCATAACACACCGCGGCTAATGCAAATATTTTCGTGAAAAGAT |
| | CTTCCATTACTCGAAGTTTTAAAACTAAAAACAAAATATTATTTCAAA |
| | AGATCATGAAGAAATTTCTGTCGGTAACAGCAGTTTCAATTGATAAA |
| | TCACCATCACTTATCTAGGAGGTTCTGTTACTCTAGAAGTGAATTAAG |
| | CAGGACAGCTGT (SEQ ID NO: 14) |
| mU2 | Gggcggggcatgcaaataactgctctgtggaactctgggagcaaaaacaaaaaactgcaaccaaaacttctcggc |
| | ctccttgaatcttacaggcttttcgtggcgtaaaggtggtgtactcaatgaagaggagagtctgtgttggctgcatgttt |
| | gagtcggttggttggtgactgtgaatTAAAGGTGTGGtcggtgttgagtgtatggggcgtgtgggcgtagt |
| | tcggt (SEQ ID NO: 15) |
| mU6 | Cgacgccgccatctctaggcccgcgccggccccctcgcacagacttgtgggagaagctcggctactcccctgcc |
| | ccggttaatttgcatataatatttcctagtaactatagaggcttaatgtgcgataaaagacagataatctgttctttttaata |
| | ctagctacattttacatgataggcttggatttctataagagatacaaatactaaattattattttaaaaaacagcacaaaa |
| | ggaaactcaccctaactgtaaagtaattgtgtgttttgagactataaatatcccttggagaaaagccttgttt (SEQ |
| | ID NO: 16) |
| mH1 | CATgCAAATTACGCGCTgTGCTTTGTGGGAAATCACCCTAAACGTAAA |
| | ATTTATTCCTCTTTcGAGCCTTATAGTGGcGGCCGGTCTACACCCTAAA |
| | (SEQ ID NO: 17) |

In some embodiments, a human U7 promoter can comprise SEQ ID NO: 1. In some embodiments, a human U7 promoter can comprise SEQ ID NO: 2. In some embodiments, a human U1 promoter can comprise SEQ ID NO: 3. In some embodiments, a human U1 promoter can comprise SEQ ID NO: 3. In some embodiments, a human U1 promoter can comprise SEQ ID NO: 4. In some embodiments, a human U2 promoter can comprise SEQ ID NO: 5. In some embodiments, a human U4 promoter can comprise SEQ ID NO: 6. In some embodiments, a human U5 promoter can comprise SEQ ID NO: 7. In some embodiments, a human U6 promoter can comprise SEQ ID NO: 8. In some embodiments, an h7sk promoter can comprise SEQ ID NO: 9. In some embodiments, a tRNA (val) promoter can comprise SEQ ID NO: 10.

In some embodiments, a murine U1 promoter can comprise SEQ ID NO: 11. In some embodiments, a murine U7 promoter can comprise SEQ ID NO: 12. In some embodiments, a murine U7 promoter can comprise SEQ ID NO: 13. In some embodiments, a murine U5 promoter can comprise SEQ ID NO: 14. In some embodiments, a murine U2 promoter can comprise SEQ ID NO: 15. In some embodiments, a murine U6 promoter can comprise SEQ ID NO: 16. In some embodiments, a murine H1 promoter can comprise SEQ ID NO: 17.

### Protein Coding Promoter Sequences

In some aspects, rAAV vectors of the disclosure can be used to express one or more transgenes encoding proteins. In these aspects, the promoters are promoters suitable for the recruitment of PolII or PolIII. In some aspects, any promoter capable of regulating the expression of a protein encoding transgene may be used. In some embodiments, a promoter can include but is not limited to, the phosphoglycerate kinase (PKG) promoter, CAG (composite of the CMV enhancer the chicken beta actin promoter (CBA) and the rabbit beta globin intron), NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerate kinase promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter 101451 In some embodiments, an exemplary promoter sequence is a cytomegalovirus (CMV) promoter. In some embodiments, a CMV promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 18.

### Terminator Sequences

The rAAV vectors disclosed herein comprise a downstream terminator (DT). Downstream terminators define the end of a transcriptional unit, such as an esnRNA, snRNA, sgRNA, or transgene. In some aspects, the terminator is an snRNA terminator. In some aspects, the terminator is a noncoding RNA terminator. In some aspects, the terminator is a polyadenylation (polyA) sequence.

In one embodiment, rAAV vectors of the disclosure comprise one or more snRNAs, one or more promoters, and one or more DT. In one aspect, promoter and DT sequences provided herein may be mixed and matched in any combination.

In some embodiments, the DT comprises, consists essentially of, or consists of a nucleic acid sequence at least 65%, 70%, 75%, 80%, 85%, 90%, 95%, 96%, 97%, 98%, 99% or 100% (or any percentage in between) identical to a DT sequence listed in the Table, which follows:

| Exemplary | |
|---|---|
| DT | DT Sequence |
| hU1 | ACTTTCTGGAGTTTCAAAAACAGACTGTACGCCAAGGGTCATATCTT |
| | TTT (SEQ ID NO: 21) |
| hU2 | CCGGGGATACAACGTGTTTCCTAAAAGTAGAGGGAGGTAAGAGACG |
| | GTAG (SEQ ID NO: 22) |
| hU4 | CTGAATTTTCTTGCAGTTGAACAACAGAGGCTTTTTTTGTGTGTGTGG |
| | GG (SEQ ID NO: 23) |
| hU5 | ATATGTGGTAATCCAACAATAGAAATTATTTTTAAGTTTGTGTGTTCC |
| | TT (SEQ ID NO: 24) |
| hU6 | TTTTTT (SEQ ID NO: 25) |
| h7sk | TTTTTT (SEQ ID NO: 26) |
| tRNA(Val) | TTTTTT (SEQ ID NO: 27) |
| mU1 | GTTTACTTGGTTTTAAAAATAGCTTGCACTAGCGATACGGAATATGG |
| | TTATTAGGTTTGTTAGGCATCATGTCGTGTCTTACTATAGAAAAATA |
| | ACGTAGTGTTCATTTTAGCCTGCCTGTATGTGTTAATTTGTCCTTATT |
| | GCGCATTGTTCTTGTTAAGTCTTCTGTAAGGAGTTGCGGGTTTCAAA |
| | CTGTCAGTCTGAGAGCA (SEQ ID NO: 28) |
| mU7+ | GTTTACTTGGTTTTAAAAATAGCTTGCACTAGCGATACGGAATATGG |
| | TTATTAGGTTTGTTAGGCATCATGTCGTGTCTTACTATAGAAAAATA |
| | ACGTAGTGTTCATTTTAGCCTGCCTGTATGTGTTAATTTGTCCTTATT |
| | GCGCATTGTTCTTGTTAAGTCTTCTGTAAGGAGTTGCGGGTTTCAAA |
| | CTGTCAGTCTGAGAGCA (SEQ ID NO: 29) |
| mU7 | Cccaatttcactggtctacaatgaaagcaaaacagttctcttccccgctccccggtgtgtgagaggggctttgatcc |
| | ttctctggtttcctaggaaacgcgtatgtgtac |
| | SEQ ID NO: 30) |
| hU7 | CCTCTTATGATGTTTGTTGCCAATGATAGATTGTTTTCACTGTGCAAA |
| | AATTATGGGTAGTTTTGGTGGTCTTGATGCAGTTGTAAGCTTGGAG |
| | SEQ ID NO: 20) |
| hU7 | CTTATGATGTTTGTTGCCAATGATAGATTGTTTTCACTGTGCAAAAAT |
| | TATGGGTAGTTTTGGTGGTCTTGATGCAGTTGTAAGCTTGGAG (SEQ ID NO: 31) |
| mU5 | CCACATTTTGTGTTTAAAAATAGAAATATTTAAGTGAGATCAGTTTA |
| | AATCTGCTTTATCTAGGGTGTCTAACTGCTTGCATCTTTTTAACTTTT |
| | CCTTATTTGTGAGAAGGTCTGTAAAACTTATTATATGTTAGTACACT |
| | GTAGCTGTCTTCAGACACTCCAGAAGAGGGATTCAGATCACCTTACA |
| | GATGGTTTTGA (SEQ ID NO: 32) |
| mU2 | CCCTCTGGGGAgtaaagttggttttaaagtcagagcatggtgattgtagggcagtccaacttttttaaatat |
| | gctgtg (SEQ ID NO: 33) |
| mU6 | TTTTTT |
| mH1 | TTTTTT |

In some embodiments, the human U7 DT comprises the sequence set forth in SEQ ID NO: 20. In some embodiments, the human U1 DT comprises the sequence set forth in SEQ ID NO. 21. In some embodiments, the human U2 DT comprises the sequence set forth in SEQ ID NO: 22. In some embodiments, the human U4 DT comprises the sequence set forth in SEQ ID NO: 23. In some embodiments, the human U5 DT comprises the sequence set forth in SEQ ID NO: 24. In some embodiments, the human U6 DT comprises the sequence set forth in SEQ ID NO.; 25. In some embodiments, the human h7sk DT comprises the sequence set forth in SEQ ID NO: 26. In some embodiments, the human tRNA(val) DT comprises the sequence set forth in SEQ ID NO: 27. In some embodiments, the murine U1 DT comprises the sequence set forth in SEQ ID NO: 28. In some embodiments, the murine U7 DT comprises the sequence set forth in SEQ ID NO: 29. In some embodiments, the murine U7 DT comprises the sequence set forth in SEQ ID NO: 30. In some embodiments, the human U7 DT comprises the sequence set forth in SEQ ID NO: 31. In some embodiments, the murine U5 DT comprises the sequence set forth in SEQ ID NO: 32. In some embodiments, the murine U2 DT comprises the sequence set forth in SEQ ID NO: 33. In some embodiments, the murine U6 DT comprises TTTTTT. In some embodiments, the murine UH1 DT comprises TTTTTT.

### Recombinant AAV Vectors Buffer Sequences

In some embodiments, the rAAV vector comprises multiple nucleic acid elements such as snRNA, sgRNA, or transgenes. In some embodiments, the multiple nucleic acid elements are multiple copies of the same nucleic acid sequence. In some embodiments, the multiple copies of snRNA comprise different snRNA molecules. In some embodiments, the multiple nucleic acid elements comprise 2, 3, 4, 5, 6, 7, 8, 9, or 10 copies (2×, 3×, 4×, 5×, 6×, 7×, 8×, 9× or 10×) of the nucleic acid element. In some embodiments, the multiple nucleic acid elements are 4 or more copies of the nucleic acid elements. In some aspects, one or more nucleic acid element are identical. In some aspects, one or more nucleic acid element are different.

In some embodiments, the rAAV vector comprises multiple transgenes. In embodiments, the multiple transgenes are the same transgene. In some embodiments, the transgenes comprise different transgenes. In some embodiments, the multiple transgenes are 2, 3, 4, 5, 6, 7, 8, 9, or 10 (2×, 3×, 4×, 5×, 6×, 7×, 8×, 9× or 10×) transgenes. In some embodiments, the transgenes are 4 or more copies of the transgene. In some aspects, one or more transgene are identical. In some aspects, one or more transgene are different.

In some embodiments, the rAAV vector comprises multiple copies of snRNA. In embodiments, the multiple copies are the same snRNA. In some embodiments, the multiple copies of snRNA comprise different snRNA molecules. In some embodiments, the multiple copies of the snRNA are 2, 3, 4, 5, 6, 7, 8, 9, or 10 copies (2×, 3×, 4×, 5×, 6×, 7×, 8×, 9× or 10×) of the snRNA. In some embodiments, the multiple copies of the snRNA are 4 or more copies of the snRNA. In some aspects, one or more snRNA are identical. In some aspects, one or more snRNA are different.

In some aspects, each nucleic acid element of the nucleic acid elements Are separated by a nucleic acid buffer sequence derived from human non-coding genomic sequences. In some aspects, each snRNA of the multiple copies of snRNA is separated by a nucleic acid buffer sequence derived from human non-coding genomic sequences downstream of an snRNA. In one embodiment, the buffer sequence is derived from human genomic sequences downstream of U7.

In one embodiment, the buffer sequence is one of the following nucleic acid sequences:

buffer 1 (30 bp)
(SEQ ID NO: 98)
CAAACTACAGAGCCAAGTGCTATCCACAGA,
buffer 2 (30 bp)
(SEQ ID NO: 99)
GAGCTTTCTGGGTTGCCATCTCAAGCAGAC,
buffer 3 (30 bp)
(SEQ ID NO: 100)
TACAAGGCCATCAGCTCATACTCACAATTG,
or a combination thereof.

In another embodiment, the buffer sequence is one of the following nucleic acid sequences:

buffer 1 (100 bp)
(SEQ ID NO: 101)
CAAACTACAGAGCCAAGTGCTATCCACAGAGAGCTTTCTGGGTTGCCAT
CTCAAGCAGACTACAAGGCCATCAGCTCATACTCACAATTGACTTTGAG
AG,
buffer 2 (100 bp)
(SEQ ID NO: 102)
TTGACCACATACGTGCTCTTTCAAAGTTCTGTGTTTGAAGTTATGTTAG
TAACAACTGATGCCCATCCTGCAATGACAAATCCAATTCTCAGTGCAGC
TC,
or a combination thereof.

In another embodiment, the buffer sequence is one of the following nucleic acid sequence:

buffer 1 (500 bp)
(SEQ ID NO: 103)
CAAACTACAGAGCCAAGTGCTATCCACAGAGAGCTTTCTGGGTTGCCAT
CTCAAGCAGACTACAAGGCCATCAGCTCATACTCACAATTGACTTTGAG
AGTCATTTTCCAATGCTCCTACACACCCCTTCTTCACAATCCCCAACAA
ATCTGAGGCTGGAACTTGGTACCATAACAATCATTACATTATTTCACCA
GAAGTACACCTTGCCTGGAAGATTGGCATTATAGCATCTTCTAACATTG
TGAAAGTTAGTGACCAATGAGGAGATCCAAGTCAGTTCCAGTTGGATTT
CTCTATACTCTATAATAAATATATATGGTGTCTTCAACAATAGGACTTT
GCCATCCAGTGATGCTAAAAATCAATAACAATGGCAATAACCTGCCCTG
TTTGGAAAGCCTCTGGCTTCCATGACTAACAATTCAAGGCAGGTCTCCT
ATACCTAGTACTGAGATTTTTATTTGATAAACTATATCTTCTGGGAGGA
GAAGCATTGT,
buffer 2 (500 bp)
(SEQ ID NO: 104)
TTGACCACATACGTGCTCTTTCAAAGTTCTGTGTTTGAAGTTATGTTAG
TAACAACTGATGCCCATCCTGCAATGACAAATCCAATTCTCAGTGCAGC
TCTCTGAAATAGTTTTGCTTTCTCTCTCTAGGTCTGTTCTATACTCCTA
ACTCTCCAGGAGTTTACAAGGAATAAAATCTCTTCCAAATGCTTTCTGT
TGCAACAACTGGACCATACTGAAAGCTGAGGCCCACAATTGCAATCTAG
GTTAGCAGGTAATCATTGTTGGTGAGGTCCTCCCTTTCCCCAGGCTCGT
GTTTGTATTGGGGAGCAGGAAATTTTTGCTAGAGCAGCACTGCCATCTC
TCTACACTCCACCTGATTGGTGGGATGGACCAGAGAAATGGACATTCCC
AACACAGTCCCTCCTTTCACATCTGCTCACCTGCCCACAGGATACTTTC
CACCATGCATACTGGGCTCTGCACCAACCATTCAGCAGTGATGAAGAGG
AAACTTGAAC,
or a combination thereof.

The 100 bp and 500 bp buffer sequences are derived from a sequence starting 100 bp downstream of the Mus musculus U7 pseudogene 8 (Location Chromosome 14: 4,409,359-4,409,421 reverse strand. GRCm39:CM001007.3). The 100 bp and 500 bp buffer 2s are derived from the sequence starting 130 bp downstream of human U7 pseudogene 5 (Chromosome X: 140,451,148-140,451,208 forward strand. GRCh38:CM000685.2). Both 100 bp buffers are the first 100 bp of the corresponding 500 bp buffer. The 30 bp buffers 1, 2, and 3, are sequential 30 bp sequences within “100 bp buffer 1”, downstream of the Mus musculus U7 pseudogene 8. These downstream sequences were selected due to the lack of any known regulatory sites or genes within or nearby to the sequence (using Gencode/Ensembl), in addition to lack of repetitive sequence, 40-60% GC content for total buffer, 40-60% GC content in the 20 bp region at both ends of the buffer, and minimal sequence complexity.

snRNA Sequences

Exemplary snRNA sequences of the disclosure can comprise any combination of snRNA features, including engineered snRNA feature sequences, described herein. In some aspects, the snRNA (or esnRNA) comprises, consists essentially of, or consists of a nucleic acid sequence at least 65%, 70%, 75%, 80%, 85%, 90%, 95%, 96%, 97%, 98%, 99% or 100% (or any percentage in between) identical to snRNA set forth in any one of SEQ ID NO: 34-SEQ ID NO: 40.

In some embodiments, an snRNA of the disclosure comprises SEQ ID NO: 34. In some embodiments, an snRNA of the disclosure comprises SEQ ID NO: 35. In some embodiments, an snRNA of the disclosure comprises SEQ ID NO: 36. In some embodiments, an snRNA of the disclosure comprises SEQ ID NO: 37. In some embodiments, an snRNA of the disclosure comprises SEQ ID NO: 38. In some embodiments, an snRNA of the disclosure comprises SEQ ID NO: 39. In some embodiments, an snRNA of the disclosure comprises SEQ ID NO: 40.

### Vectors

Within the context of a recombinant expression vector, the terminology “operably linked” is intended to mean that a promoter is linked to a nucleotide sequence of interest (NOI), such as a nucleic acid element described herein including sgRNA or transgenes, in a manner permitting expression of the nucleotide sequence in, for example, a host cell when the vector is introduced into (or in contact with) the host cell.

In some embodiments of the compositions and methods of the disclosure, a vector comprises one or more promoter each regulating expression of one or more nucleic acid sequence such as a noncoding RNA or protein encoding transgene. In some embodiments, the vector is a single or unitary vector.

In some embodiments of the compositions and methods of the disclosure, the noncoding RNA, such as snRNA or sgRNA, are capable of targeting toxic CAG, CUG, GGCCCC, CCGGG, or GGCCC+CCGGGG RNA repeats (or flanking sequences thereof) are in a single vector. In some embodiments of the compositions and methods of the disclosure, the RNA-targeting systems are capable of targeting a non-repeat RNA of interest. In some aspects, the vectors of the disclosure are capable of targeting multiple (i.e., two or more) RNAs of interest. In some aspects, the vectors of the disclosure are capable of targeting multiple sequences within a single target RNA of interest.

One type of vector is a “plasmid”, which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. In some embodiments, the vector is a lentivirus (such as an integration-deficient lentiviral vector) or adeno-associated viral (AAV) vector. Vectors may be capable of autonomous replication in a host cell into which they are introduced such as e.g., bacterial vectors having a bacterial origin of replication and episomal mammalian vectors and other vectors such as, e.g., non-episomal mammalian vectors, are integrated into the genome of a host cell upon introduction into the host cell, and thereby are replicated along with the host genome.

In some embodiments, vectors such as e.g., expression vectors, are capable of directing the expression of genes to which they are operatively-linked. Common expression vectors are often in the form of plasmids. In some embodiments, recombinant expression vectors comprise a nucleic acid provided herein such as e.g., an snRNA in a form suitable for expression in a host cell. Recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence such as e.g., in an in vitro transcription/translation system or in a host cell when the vector is introduced into the host cell. In some embodiments, the regulatory element is a promoter described herein. In some embodiments, the regulatory element is a terminator provided herein.

Certain embodiments of a vector depend on factors such as the choice of the host cell to be transformed, and the level of expression desired. A vector can be introduced into host cells to thereby produce transcripts, proteins, or peptides, including fusion proteins or peptides, encoded by nucleic acids as described herein such as, e.g., snRNAs, CRISPR transcripts, proteins, enzymes, mutant forms thereof, fusion proteins thereof, etc.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. An “enhancer” is a region of DNA that can be bound by activating proteins to increase the likelihood or frequency of transcription.

In some embodiments of the compositions and methods of the disclosure, an expression vector, viral vector or non-viral vector provided herein, includes without limitation, vector elements such as a buffer sequence derived human genomic sequences downstream from an snRNA and as such will have the capability to encoding multiple snRNAs from a single construct.

In some embodiments, the snRNA constructs disclosed herein comprise bidirectional snRNA promoters to express snRNAs.

In another embodiment, the vector configurations can comprise linker(s), signal sequence(s), and/or tag(s).

### Viral Vectors

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors.

In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis.

In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

### Lentiviral Vectors

Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIVSM) vector, a modified sooty mangabey simian immunodeficiency virus (SIVSM) vector, a African green monkey simian immunodeficiency virus (SIVAGM) vector, a modified African green monkey simian immunodeficiency virus (SIVAGM) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/MV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Adeno-Associated Virus Vectors

In some aspect, a vector described herein is an AAV viral vector. The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3: 1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control. Multiple serotypes of this virus are known to be suitable for gene delivery; all known serotypes can infect cells from various tissue types.

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928. U.S. Pat. No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome.

The cap gene is expressed from the p40 promoter and encodes the three capsid proteins, VP1, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs. a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity”. Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VRI-IX also referred to as VR1-VR8). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA to generate AAV vectors. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV vectors

An “rAAV vector” as used herein refers to a vector comprising, consisting essentially of, or consisting of one or more nucleic acid elements described herein and one or more AAV inverted terminal repeat sequences (ITRs). Such AAV vectors can be replicated and packaged into infectious viral particles, comprising AAV capsid proteins of the disclosure, when present in a host cell that provides the functionality of rep and cap gene products: for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing rAAV vectors may also contain elements for manufacturing purposes, e.g., antibiotic resistance genes, origin of replication sequences etc., but these are not encapsidated and thus do not form part of the AAV particle.

In some aspects, an rAAV vector can comprise at least two promoter sequences operably linked to nucleic acid elements such as a noncoding RNA or transgene. In some aspects, an rAAV vector can comprise at least one AAV inverted terminal (ITR) sequence. In some aspects, an rAAV vector can comprise at least one promoter sequence. In some aspects, an rAAV vector can comprise at least one enhancer sequence. In some aspects, an rAAV vector can comprise at least one polyA sequence. In some aspects, an rAAV vector can comprise at least one reporter protein.

In some aspects, an rAAV vector can comprise more than one nucleic acid elements such as a transgene nucleic acid molecule or more than one noncoding RNA molecule. In some aspects, an rAAV vector can comprise at least two transgene nucleic acid molecules or at least two noncoding RNA molecules, such that the rAAV vector comprises a first transgene nucleic acid molecule or noncoding RNA molecule and an at least a second transgene nucleic acid molecule or noncoding RNA molecule. In some aspects, the first and the at least second transgene nucleic acid molecule or noncoding RNA molecule can comprise the same nucleic acid sequence. In some aspects, the first and the at least second transgene nucleic acid molecules or noncoding RNA molecules can comprise different nucleic acid sequences. In some aspects, the rAAV vector comprises at least two, at least three, at least four, at least five, at least six, at least seven, at least eight, or more nucleic acid elements (including noncoding RNA or transgenes) wherein each nucleic acid element is operably linked to a promoter. In some aspects, the promoter operably linked to each nucleic acid element is unique and/or low sequence homology relative to other promoters within the vector.

In some aspects, an rAAV vector can comprise more than one promoter sequence. In some aspects, an rAAV vector can comprise at least two promoter sequences, such that the rAAV vector comprises a first promoter sequence and an at least second promoter sequence. In some aspects, the first and the at least second promoter sequences can comprise the same sequence. In some aspects, the first and the at least second promoter sequences can comprise different sequences. In some aspects, the first and the at least second promoter sequences can be adjacent to each other. In some aspects wherein an rAAV vector also comprises a first transgene nucleic acid molecule or noncoding RNA molecule and an at least second transgene nucleic acid molecule or noncoding RNA molecule, the first promoter can be located upstream (5′) of the first transgene nucleic acid molecule and the at least second promoter can be located between the first transgene nucleic acid molecule or noncoding RNA molecule and the at least second transgene nucleic acid molecule or noncoding RNA molecule, such that the at least second promoter is downstream (3′) of the first transgene nucleic acid molecule or noncoding RNA molecule and upstream (5′) of the at least second transgene nucleic acid molecule or noncoding RNA molecule.

Any of the preceding rAAV vectors can further comprise at least one enhancer. The at least one enhancer can be located anywhere in the rAAV vector. In some aspects, the at least one enhancer can be located immediately upstream (5′) of a promoter.

rAAV vectors of the disclosure can comprise any nucleic acid element of the disclosure including but not limited to: transgene sequences encoding for proteins and/or peptides, noncoding RNA, RNA binding noncoding RNA, small-nuclear RNA (snRNA) molecule, a single guide RNA molecule (sgRNA), a microRNA, a short hairpin RNA (shRNA), an enhancer RNA (eRNA), a small nucleolar RNA (snoRNA), or a long noncoding RNA (lncRNA). In some aspects, the sgRNA is used in conjunction with CRISPR/Cas systems to target, bind, and/or cleave nucleic acids including DNA and RNA sequences. In some aspects, a transgene nucleic acid molecule is referred to interchangeably as a nucleotide sequence of interest (NOI). An NOI includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region, but need not be a coding region. An NOI can be RNA/DNA in a sense or anti-sense orientation. NOIs are also referred herein, without limitation, as transgenes, heterologous sequences, genes, therapeutic genes. An NOI may also encode a POI (protein of interest), a partial POI, a mutated version or variant of a POI. A POI may be analogous to or correspond to a wild-type protein. A POI may also be a fusion protein or nucleoprotein complex such as a CRISPR/Cas nucleoprotein complex. A POI may also be a PUF or PUMBY protein. In some aspects, POIs can be RNA targeting or RNA-binding proteins or nucleoprotein complexes. In some aspects, the NOI is a noncoding RNA molecule such as an snRNA or sgRNA.

Recombinant AAV (rAAV) genomes of the invention may comprise, consist essentially of, or consist of one or more nucleic acid elements and one or more AAV ITRs flanking the nucleic acid molecule. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014). The nucleotide sequences of the genomes of various AAV serotypes are known in the art.

An AAV vector described herein may comprise, consist essentially of, or consist of one or more nucleic acid molecules and one or more AAV ITRs. In some aspects, the nucleic acid molecule encodes an snRNA of the disclosure. Such AAV vectors can be replicated and packaged into infectious viral particles when present in a host cell that provides the functionality of rep and cap gene products, for example, by transfection of the host cell. In some aspects, AAV vectors contain a promoter, at least one nucleic acid that may encode at least one protein or RNA, and/or an enhancer and/or a terminator within the flanking ITRs that is packaged into the infectious AAV particle. The encapsidated nucleic acid portion may be referred to as the AAV vector genome. Plasmids containing AAV vectors may also contain elements for manufacturing purposes, e.g., antibiotic resistance genes, origin of replication sequences etc., but these are not encapsidated and thus do not form part of the AAV particle.

In some aspects, an AAV vector can comprise at least one nucleic acid element of the disclosure. In some aspects, an AAV vector can comprise at least one regulatory sequence. In some aspects, an AAV vector can comprise at least one AAV inverted terminal (ITR) sequence. In some aspects, an AAV vector can comprise a first ITR sequence and a second ITR sequence. In some aspects, an AAV vector can comprise at least one promoter sequence. In some aspects, an AAV vector can comprise at least one enhancer sequence. In some aspects, an AAV vector can comprise at least one terminator sequence. In some aspects, an AAV vector can comprise at least one polyA sequence. In some aspects, an AAV vector can comprise at least one linker sequence. In some aspects, an AAV vector can comprise at least one buffer sequence. In some aspects, an AAV vector of the disclosure can comprise at least one nuclear localization signal, or nuclear export signal and/or both.

In some aspects, an AAV vector can comprise a first AAV ITR sequence, a promoter sequence, an snRNA sequence, a terminator sequence and a second AAV ITR sequence. In some aspects, an AAV vector can comprise, in the 5′ to 3′ direction, a first AAV ITR sequence, a promoter sequence, an snRNA sequence, a terminator sequence, and a second AAV ITR sequence.

In some aspects, an AAV vector can comprise a first AAV ITR sequence, a first promoter sequence, a first nucleic acid element sequence, a termination sequence, a second promoter sequence, second nucleic acid element sequence, a second termination sequence and a second AAV ITR sequence. In some aspects, an AAV vector can comprise a first AAV ITR sequence, a first promoter sequence, a first nucleic acid element sequence, a termination sequence, a second promoter sequence, a second nucleic acid element sequence, a second termination sequence, a third promoter sequence, a third nucleic acid element sequence, a third termination sequence, and a second AAV ITR sequence. In some aspects, an AAV vector can comprise a first AAV ITR sequence, a first promoter sequence, a first nucleic acid element sequence, a termination, a second promoter sequence, a second nucleic acid element sequence, a second termination sequence, a third promoter sequence, a third nucleic acid element sequence, a third termination sequence, a fourth promoter sequence, a fourth nucleic acid element sequence, a fourth termination sequence, and a second AAV ITR sequence.

In some aspects, an AAV vector can comprise a first AAV ITR sequence, a first promoter sequence, a first snRNA sequence, a termination sequence, a second promoter sequence, second snRNA sequence, a second termination sequence and a second AAV ITR sequence. In some aspects, an AAV vector can comprise a first AAV ITR sequence, a first promoter sequence, a first snRNA sequence, a termination sequence, a second promoter sequence, a second snRNA sequence, a second termination sequence, a third promoter sequence, a third snRNA sequence, a third termination sequence, and a second AAV ITR sequence. In some aspects, an AAV vector can comprise a first AAV ITR sequence, a first promoter sequence, a first snRNA sequence, a termination, a second promoter sequence, a second snRNA sequence, a second termination sequence, a third promoter sequence, a third snRNA sequence, a third termination sequence, a fourth promoter sequence, a fourth snRNA sequence, a fourth termination sequence, and a second AAV ITR sequence.

In some embodiments of the compositions and methods of the disclosure, the viral vector comprises a sequence isolated or derived from an adeno-associated virus (AAV). In some embodiments, the viral vector comprises an ITR sequence or a capsid sequence that is isolated or derived from an AAV of serotype AAV1, AAV2, AAV3, AAV3B, AAV4, AAV5, AAV6, AAV7, AAV8, AAV9, AAV10, AAV11, AAV12, AAV13, AAVPO1, AAVPHP.B, AAVrh74 or AAVrh.10. In some embodiments, the AAV serotype is AAVrh.74. In one embodiment, the AAV vector comprises a modified capsid. In one embodiment the AAV vector is an AAV2-Tyr mutant vector. In one embodiment the AAV vector comprises a capsid with a non-tyrosine amino acid at a position that corresponds to a surface-exposed tyrosine residue in position Tyr252, Tyr272, Tyr275, Tyr281, Tyr508, Tyr612, Tyr704, Tyr720, Tyr730 or Tyr673 of wild-type AAV2. See also WO 2008/124724 incorporated herein in its entirety. In some embodiments, the AAV vector comprises an engineered capsid. AAV vectors comprising engineered capsids include without limitation, AAV2.7m8, AAV9.7m8, AAV2 2tYF, and AAV8 Y733F). In some embodiments, the capsid is a ubiquitination resistant capsid. In another embodiment, the ubiquitination capsid is an AAV2 capsid comprising tyrosine (Y) and serine (S) mutations. In another embodiment, the AAV2 capsid comprises Y, S and threonine (T) mutations. In another embodiment, the AAV2 capsid includes, without limitation, AAV2 capsid mutants such as T455V, T491V, T550V, T659V, Y444+500+730F, and Y444+500+730F+T491V. In some embodiments, the viral vector is replication incompetent. In some embodiments, the viral vector is isolated or recombinant (rAAV). In some embodiments, the viral vector is self-complementary (scAAV). In some embodiments, the viral vector is single-stranded (ssAAV).

In some embodiments, the snRNAs provided herein are comprised within a single-stranded AAV (ssAAV). In some embodiments, the snRNAs provided herein are comprised within a self-complementary AAV (scAAV). The single-stranded nature of the parvoviral genome requires the use of cellular mechanisms to provide a complementary-strand for gene expression. This cellular recruitment activity is considered a rate-limiting factor in the efficiency of transduction and gene expression in parvoviruses and parvoviral particles. The use of an scAAV versus an ssAAV remedies this well-known issue by packaging both strands as a single duplex DNA molecule (or inverted repeat genome) that can fold into dsDNA as a result of a self-complementary viral genome sequence. In this regard, the requirement for DNA synthesis or base-pairing between multiple viral genomes is eliminated.

### AAV ITR Sequences

In some embodiments of the compositions and methods of the disclosure, an AAV inverted terminal repeat sequence can comprise any AAV ITR sequence known in the art. In some aspects, an AAV ITR sequence can comprise or consist of an AAV1 ITR sequence, an AAV2 ITR sequence, an AAV3 ITR sequence, an AAV4 ITR sequence, an AAV5 ITR sequence, an AAV6 ITR sequence, an AAV7 ITR sequence, an AAV8 ITR sequence, an AAV9 ITR sequence, an AAV10 ITR sequence, an AAVrh10 ITR sequence, an AAV11 ITR sequence, an AAV12 ITR sequence, an AAV13 ITR sequence, or an AAVrh74 ITR sequence.

In some aspects the ITR sequence can comprise a modified AAV ITR sequence.

In some aspects, an AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 50, 51, 52, 53, 54, or 55.

In some embodiments, an AAV vector provided herein comprises a first and a second AAV ITR sequence. In some aspects, a first AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 50, 51, 52, 53, 54, or 55 and a second AAV ITR sequence can comprise, consist essentially of, or consist of a nucleic acid sequence at least 65%, 70%, 75%, 80%, 85%, 90%, 95%, 96%, 97%, 98%, 99% or 100% (or any percentage in between) identical to SEQ ID NO: 50, 51, 52, 53, 54, or 55. In some aspects the first AAV ITR sequence is positioned at the 5′ of an AAV vector. In some aspects the second AAV ITR sequence is positioned at the 3′ of an AAV vector.

In some embodiments of the compositions and methods of the disclosure, the viral vector comprises a sequence isolated or derived from an adeno-associated virus (AAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

Exemplary snRNA Constructs of the Disclosure

Exemplary snRNA rAAV vectors of the disclosure can comprise one or more snRNA sequences of the disclosure each regulated by a distinct promoter sequence as described herein. In some aspects, the rAAV vector comprises, consists essentially of, or consists of a nucleic acid sequence at least 65%, 70%, 75%, 80%, 85%, 90%, 95%, 96%, 97%, 98%, 99% or 100% (or any percentage in between) identical to an snRNA rAAV vector listed in the snRNA rAAV vector Table which follows:

| Description | SEQ ID NO: |
|---|---|
| ssAAV U7 CMV GFP | 41 |
| ssAAV U7 U7 U7 CMV GFP | 42 |
| ssAAV U7 U1 | 43 |
| scAAV U7 U7 U7 U7 | 44 |
| scAAV U1 U7 U4 U5 (same snRNA) | 45 |
| scAAV U1 U7 U4 U5 (two distinct snRNA) | 46 |
| scAAV U1 U7 U4 U5 | 47 |
| rAAV A04384 | 48 |
| rAAV A04526 | 49 |

The ssAAV U7 CMV GFP vector is a ssAAV vector encoding an snRNA molecule operably linked to U7 promoter. The vector further encodes GFP protein operably linked to a CMV promoter. In some aspects, the ssAAV U7 CMV GFP comprises SEQ ID NO: 41. In some aspects, the vector comprises from 5′ to 3′ a first ITR sequence, a U7 promoter sequence, a nucleic acid sequence encoding an snRNA molecule, a CMV promoter, a nucleic acid sequence encoding GFP, and a second ITR sequence.

The ssAAV U7 U7 U7 CMV GFP vector is a ssAAV vector encoding three snRNA molecules each operably linked to distinct copies of a U7 promoter. The vector further encodes GFP protein operably linked to a CMV promoter. In some aspects, the ssAAV U7 U7 U7 CMV GFP vector comprises SEQ ID NO: 42. In some aspects, the vector comprises from 5′ to 3′ a first ITR sequence, a first U7 promoter sequence, a nucleic acid sequence encoding a first snRNA molecule, a second U7 promoter sequence, a nucleic acid sequence encoding a second snRNA molecule, a third U7 promoter sequence, a nucleic acid sequence encoding a third snRNA molecule, a CMV promoter, a nucleic acid sequence encoding GFP, and a second ITR sequence.

The ssAAV U7 U1 vector is a ssAAV vector encoding two snRNA molecules each operably linked to an snRNA promoter. In some aspects, the ssAAV U7 U1 vector comprises SEQ ID NO: 43. In some aspects, the vector comprises from 5′ to 3′ a first ITR sequence, a U7 promoter sequence, a nucleic acid sequence encoding a first snRNA molecule, a U1 promoter sequence, a nucleic acid sequence encoding a second snRNA molecule, and a second ITR sequence.

The scAAV U7 U7 U7 U7 vector is a scAAV vector encoding four snRNA molecules each operably linked to an snRNA promoter. In some aspects, the ssAAV U7 U7 U7 U7 vector comprises SEQ ID NO: 44. In some aspects, the vector comprises from 5′ to 3′ a first ITR sequence, a first U7 promoter sequence, a nucleic acid sequence encoding a first snRNA molecule, a second U7 promoter sequence, a nucleic acid sequence encoding a second snRNA molecule, a third U7 promoter sequence, a nucleic acid sequence encoding a third snRNA molecule, a fourth U7 promoter sequence, a nucleic acid sequence encoding a fourth snRNA molecule, and a second ITR sequence.

The scAAV U1 U7 U4 U5 vector is a scAAV vector encoding four copies of an snRNA molecule each copy operably linked to an snRNA promoter. In some aspects, the ssAAV U1 U7 U4 U5 vector comprises SEQ ID NO: 45. In some aspects, the vector comprises from 5′ to 3′ a first ITR sequence, a U1 promoter sequence, a nucleic acid sequence encoding a first snRNA molecule, a U7 promoter sequence, a nucleic acid sequence encoding a second snRNA molecule, a U4 promoter sequence, a nucleic acid sequence encoding a third snRNA molecule, a U5 promoter sequence, a nucleic acid sequence encoding a fourth snRNA molecule, and a second ITR sequence.

The scAAV U1 U7 U4 U5 vector is a scAAV vector encoding two copies of first snRNA molecule and two copies of a second snRNA molecule each copy operably linked to an snRNA promoter. In some aspects, the ssAAV U1 U7 U4 U5 vector comprises SEQ ID NO: 46. In some aspects, the vector comprises from 5′ to 3′ a first ITR sequence, a U1 promoter sequence, a nucleic acid sequence encoding a first copy of a first snRNA molecule, a U7 promoter sequence, a nucleic acid sequence encoding a first copy of a second snRNA molecule, a U4 promoter sequence, a nucleic acid sequence encoding a second copy of the second snRNA molecule, a U5 promoter sequence, a nucleic acid sequence encoding a second copy of the first snRNA molecule, and a second ITR sequence.

The vector A04384 depicts a scAAV snRNA vector comprising snRNA 38 controlled by a murine U7 promoter and snRNA 42 controlled by a human U7 promoter. In some aspects, the A04384 vector comprises SEQ ID NO: 48.

The vector A04526 depicts a scAAV snRNA expression cassette comprising snRNA 38/42 controlled by a murine U7 promoter and snRNA 38/42 controlled by a human U7 promoter. In some aspects, the A04384 vector comprises SEQ ID NO. 49.

### Nucleic Acids

An NOI (nucleotide sequence of interest) also referred to as a nucleic acid element includes, without limitation, any nucleotide sequence or transgene capable of being delivered by a vector. NOIs can be synthetic, derived from naturally occurring DNA or RNA, codon optimized, recombinant RNA/DNA, cDNA, partial genomic DNA, and/or combinations thereof. The NOI can be a coding region or partial coding region, but need not be a coding region. An NOI can be RNA/DNA in a sense or anti-sense orientation. An NOI can be an snRNA or sgRNA. NOIs are also referred herein, without limitation, as transgenes, heterologous sequences, genes, therapeutic genes. An NOI may also encode an RNA (ribonucleoprotein complex) a POI (protein of interest), a partial POI, a mutated version or variant of a POI. A POI may be analogous to or correspond to a wild-type protein. A POI may also be a fusion protein or ribonucleoprotein complex such as an snRNP.

### Cells

Also provided herein are cells comprising the RNA targeting systems, snRNA molecules and expression constructs described herein. In some aspects, the disclosure provides a cell comprising a vector, viral vector, rAAV vector or AAV viral vector of the disclosure.

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoietic stem cell (HSC).

In some embodiments of the compositions and methods of the disclosure, a somatic cell of the disclosure is a neuronal cell. In one embodiment, a cell or cells of a patient treated with compositions disclosed herein include, without limitation, central nervous system (neurons), peripheral nervous system (neurons), peripheral motor neurons, and/or sensory neurons. In one embodiment, a neuronal cell is a glial cell.

In some embodiments of the compositions and methods of the disclosure, a somatic cell of the disclosure is a fibroblast or an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the disclosure, a somatic cell is an ocular cell. An ocular cell includes, without limitation, corneal epithelial cells, keratocytes, retinal pigment epithelial (RPE) cells, lens epithelial cells, iris pigment epithelial cells, conjunctival fibroblasts, non-pigmented ciliary epithelial cells, trabecular meshwork cells, ocular choroid fibroblasts, conjunctival epithelial cells. In some embodiments, an ocular cell is a retinal cell or a corneal cell. In one embodiment, a retinal cell is a photoreceptor cell or a retinal pigment epithelial cell. In another embodiment, a retinal cell is a ganglion cell, an amacrine cell, a bipolar cell, a horizontal cell, a Müller glial cell, a rod cell, or a cone cell. In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Pharmaceutical Compositions of the Disclosure

The disclosure provides a pharmaceutical composition comprising a vector, viral vector, or AAV viral vector of the disclosure. In some aspects the vector, viral vector, or AAV viral vector comprises an rAAV viral vector of the disclosure.

### Methods of Use

The disclosure provides a method of treating a disease or disorder in a subject in need thereof comprising administering a therapeutically effective amount of a viral vector or pharmaceutical composition of the disclosure.

The disclosure provides a method of encoding an RNA or expressing an NOI in a cell using vectors and/or rAAV vectors disclosed herein. In one embodiment, the disclosure provides a method of modifying an RNA or the activity of a protein encoded by an RNA molecule comprising contacting the composition of the disclosure and the target RNA molecule under conditions suitable for binding to the RNA molecule.

The disclosure provides a method of modifying the level of expression of an RNA molecule of the disclosure or a protein encoded by the RNA molecule comprising contacting the composition of the disclosure and a cell comprising the RNA molecule under conditions suitable for binding to the RNA molecule. In some embodiments, the cell is in vivo, in vitro, ex vivo or in situ. In some embodiments, the composition of the disclosure comprises a vector comprising snRNA sequences. In some embodiments, the vector is an AAV.

The disclosure provides a method of modifying the level of expression of an RNA molecule of the disclosure or a protein encoded by the RNA molecule comprising contacting the composition of the disclosure and the RNA molecule under conditions suitable for knocking down, blocking, splicing, multi-targeting, or editing the target RNA. In some embodiments, the vector is an AAV.

The disclosure provides a method of modifying a target RNA or an activity of a protein encoded by an RNA molecule comprising contacting the composition and a cell comprising the RNA molecule under conditions suitable knocking down, blocking, splicing, multi-targeting, or editing the target RNA. In some embodiments, the cell is in vivo, in vitro, ex vivo or in situ. In some embodiments, the composition comprises a vector comprising the snRNA sequences disclosed herein. In some embodiments, the vector is an AAV.

The disclosure provides a method of treating a disease or disorder comprising administering to a subject a therapeutically effective amount of an snRNA composition of the disclosure.

The disclosure provides a method of treating a disease in a patient in need of such treatment comprising administering to the patient a therapeutically effective amount of a noncoding RNA sequence or transgene composition of the disclosure, wherein the composition comprises a vector comprising noncoding RNA sequences or transgenes disclosed herein, wherein the composition modifies, reduces, destroys, knocks down or ablates a level of expression of a toxic repeat RNA (compared to the level of expression of a toxic repeat RNA treated with a non-targeting (NT) control or compared to no treatment). In another embodiment, the level of reduction is I-fold or greater. In another embodiment, the level of reduction is 2-fold, 3-fold, 4-fold, 5-fold, 6-fold, 7-fold, 8-fold, 9-fold or 10-fold. In another embodiment, the level of reduction is 10-fold or greater. In another embodiment, the level of reduction is between 10-fold and 20-fold. In another embodiment, the level of reduction is 11-fold, 12-fold, 13-fold, 14-fold, 15-fold, 16-fold, 17-fold, 18-fold, 19-fold, or 20-fold. In another embodiment, the gene therapy compositions disclosed herein when administered to a patient lead to 20%-100% destruction of the toxic repeat RNA. In one embodiment, the % elimination of the toxic repeat RNA is any of 20-99%, 25%-99%, 50%-99%, 80%-99%, 90%-99%, 95%-99%. In one embodiment, the % elimination is 90%, 91%, 92%, 93%, 94%, 95%, 96%, 97%, 98%, or 99%. In another embodiment, % elimination is complete elimination or 100% elimination of the toxic repeat RNA.

In some embodiments of the methods of the disclosure, a subject of the disclosure has been diagnosed with a disease to be treated. In some embodiments, the subject of the disclosure presents at least one sign or symptom of a disorder or disease to be treated. In some embodiments, the subject of the disclosure presents at least one sign or symptom of a disease.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject via intracerebral administration. In some embodiments, the composition of the disclosure is administered to the subject by an intrastriatal route. In some embodiments, the composition of the disclosure is administered to the subject by a stereotaxic injection or an infusion. In some embodiments, the composition is administered to the brain. In some embodiments of the methods of the disclosure, a composition of the disclosure is administered to the subject locally.

In some embodiments, the compositions disclosed herein are formulated as pharmaceutical compositions. Briefly, pharmaceutical compositions for use as disclosed herein may comprise a protein(s) or a polynucleotide encoding the protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for routes of administration, such as e.g., oral, enteral, topical, transdermal, intranasal, and/or inhalation; and for routes of administration via injection or infusion such as, e.g., intravenous, intramuscular, subpial, intrathecal, intraparenchymal, intrathecal, intrastriatal, subcutaneous, intradermal, intraperitoneal, intratumoral, intravenous, intraocular, and/or parenteral administration. In certain embodiments, the compositions of the present disclosure are formulated for intracerebral or intrastriatal administration.

### EXAMPLES

### Example 1: Improved snRNA Expressing AAV Vectors

snRNA Expression Cassettes Having Repeated Promoter Sequences

AAV viral vectors containing snRNA expression cassettes were constructed and the expression the packaged snRNA molecules was evaluated. Initially, vectors were evaluated having one or more snRNA molecules, each under the control of a U7 promoter (FIG. 1A). A02888 depicts a single-stranded AAV (ssAAV) snRNA expression cassette comprising a single CUG repeat targeting snRNA controlled by a U7 promoter. A02896 depicts an ssAAV snRNA expression cassette comprising a three CUG repeat targeting snRNA molecules each under the control of a U7 promoter. A03624 depicts a self-complementary AAV (scAAV) snRNA expression cassette comprising from 5′ to 3′: a U7 promoter driving expression of snRNA 38, a U7 promoter driving expression of snRNA 42, a U7 promoter driving expression of snRNA 42, and a U7 promoter driving expression of snRNA 38. A03081 depicts an scAAV snRNA expression cassette comprising from 5′ to 3′: a U7 promoter driving expression of a CAG-targeting snRNA, a U7 promoter driving expression of a CAG-targeting snRNA, a U7 promoter driving expression of a CAG-targeting snRNA, and a U7 promoter driving expression of a CAG-targeting snRNA.

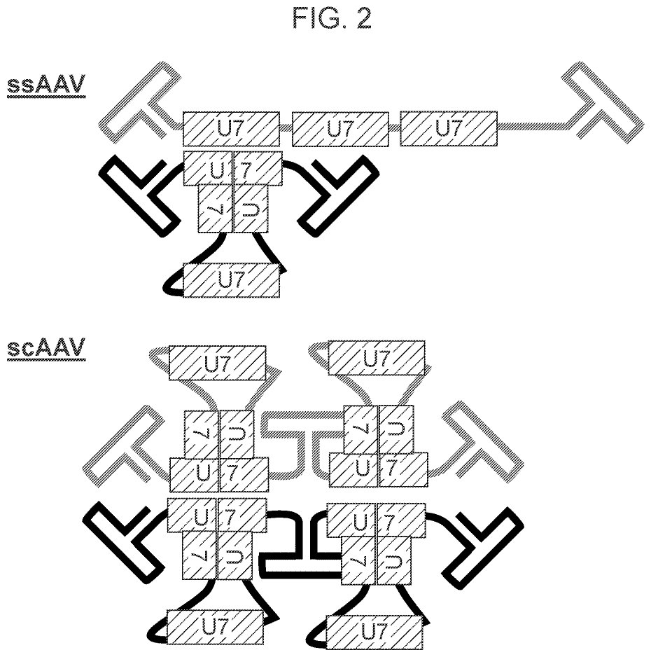

The packaging integrity of the U7 vectors was evaluated using an Agilent Tapestation. The packaging integrity (i.e. genomic integrity) measures how much intact rAAV vector is present in an AAV viral vector after encapsidation and purification and can assess the presence of any truncated products that result from homolog between discrete sections of an AAV vector such as repeated snRNA promoters or promoters having high sequence homology. For the vectors depicted in FIG. 1A, single and self-complementary vectors A029888 and A03624 contained a 1× band indicating a nucleic acid sequence having a single snRNA (FIG. 1B). For the vectors A02896 and A03081, each comprising three or four snRNA sequences, a mixture of species was observed correlating to one (1×), two (2×), three (3×) and four (4×) snRNA molecules (FIG. 1B). The 3× or 4× band was the expected species with the 1×, 2×, and 3× species indicative of truncation events. Full-length sequencing of the scAAV A03624 vector reveals multiple species corresponding to 1×, 2×, and 3× truncations (FIG. 1C). The above data demonstrates that rAAV vectors comprising snRNA expression cassettes having repeats of the same promoters can form self-complementary intramolecular or intermolecular reactions at the site of promoter repeats leading to truncations and/or other packaging issues (FIG. 2) such as conversion from a double to single stranded genome.

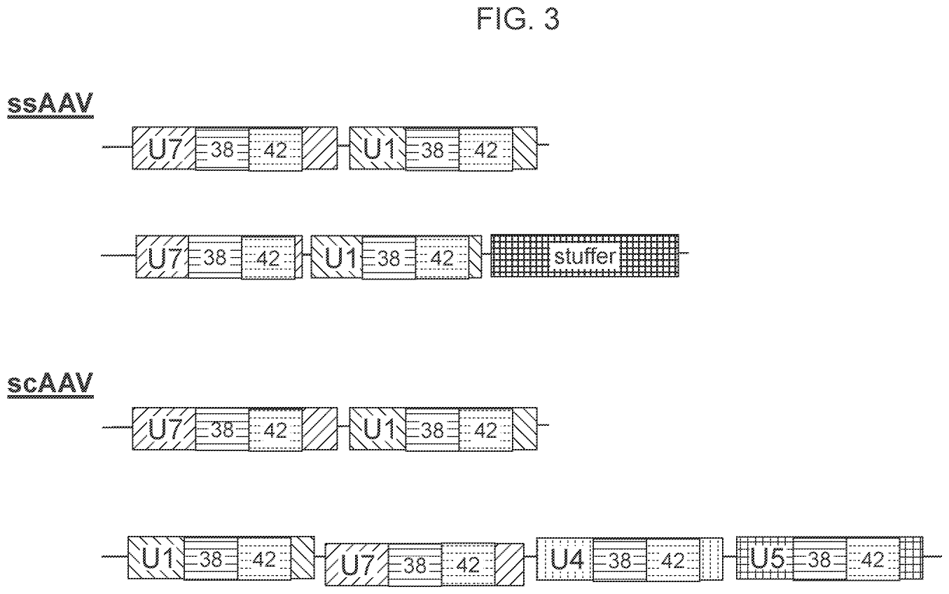

Next, AAV viral vectors having snRNA expression cassettes with varied snRNA promoters were evaluated (FIG. 3). Two ssAAV snRNA expression cassettes are depicted, the first depicting snRNA molecules, 38 and 42 under the control of a U7 promoter and a second pair of snRNAs 38 and 42 under control of a U1 promoter. A second expression cassette is depicted that further comprises a stuffer sequence. Two scAAV snRNA expression cassettes are depicted, the first depicting snRNA 38/42 under the control of a U7 promoter and a second snRNA 38/42 under control of a U1 promoter. The second scAAV expression cassette depicts from 5′ to 3′: a U1 promoter driving expression of snRNA 38/42, a U7 promoter driving expression of snRNA 38/42, a U4 promoter driving expression of snRNA 38/42, and a U5 promoter driving expression of snRNA 38/42.

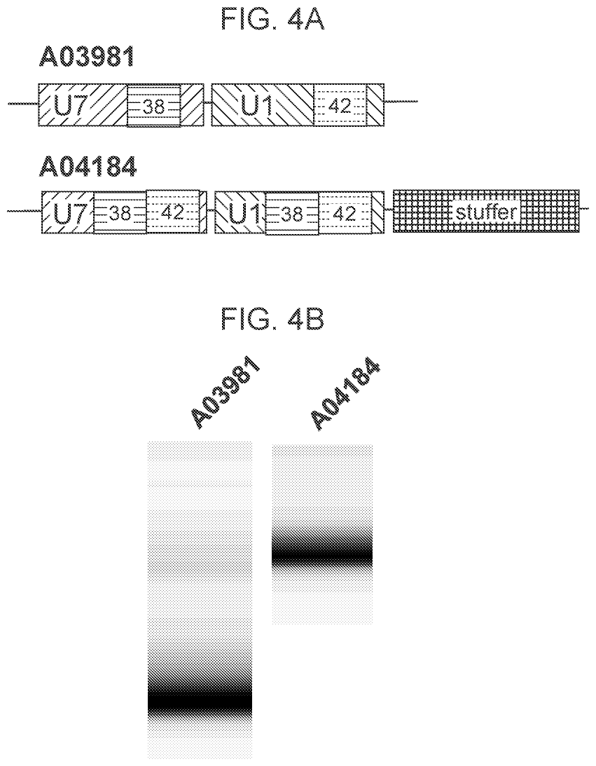

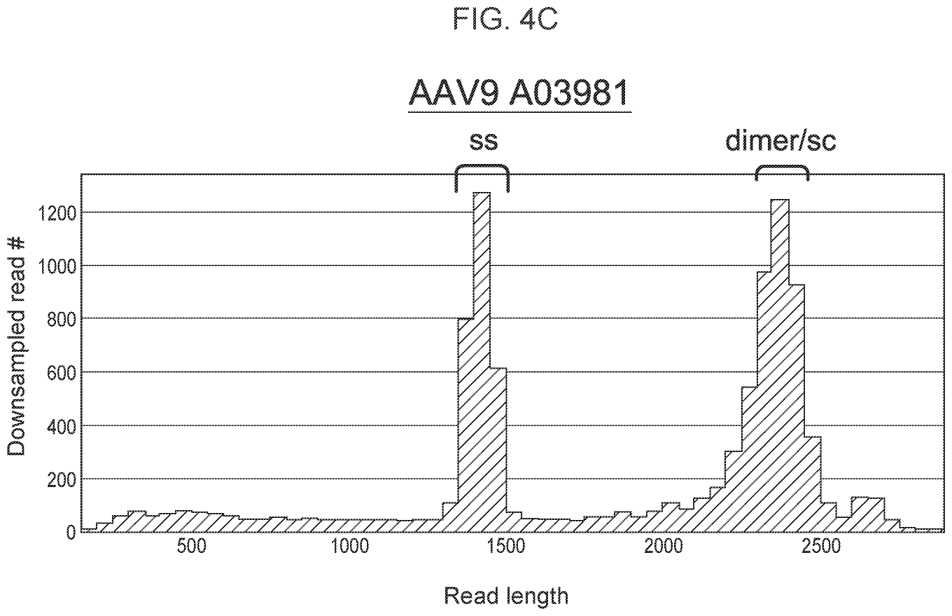

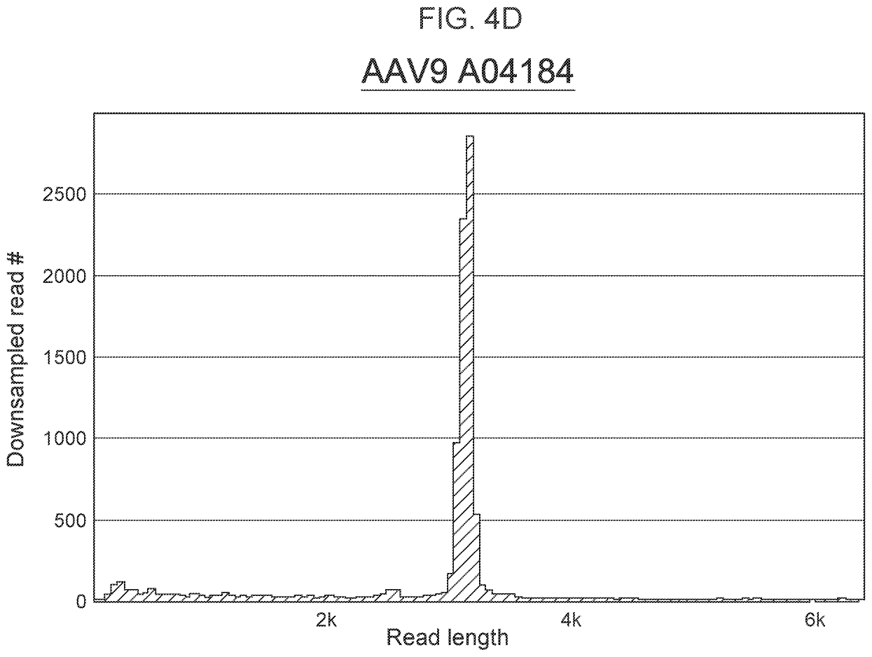

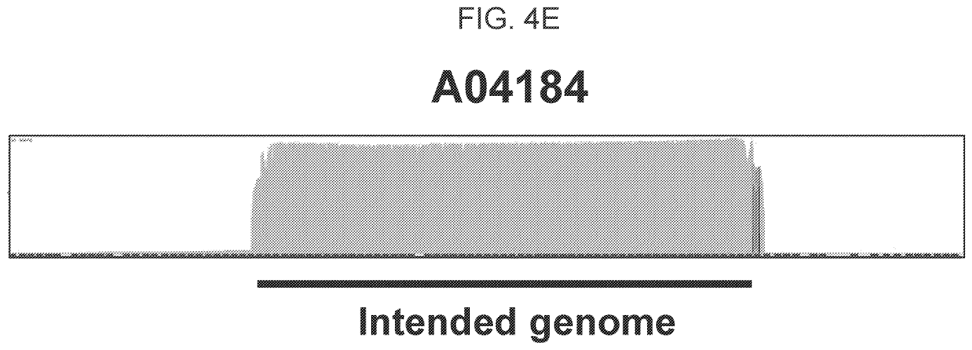

A03981 comprises snRNA molecule, 38 under the control of a U7 promoter and snRNA 42 under control of a U1 promoter (FIG. 4A). A04184 depicts snRNA 38/42 under the control of a U7 promoter and a second snRNA 38/42 under control of a U1 promoter as well as a stuffer sequence (FIG. 4A).

Following packaging and purification of AAV viral vectors containing A03981 and A04184, Tapestation images reveal a single pre-dominant species of each (FIG. 4B). Sequencing of an AAV viral vector comprising A03981 (FIG. 4C) revealed a single stranded species and a dimeric/self-complementary species. Sequencing of an AAV viral vector comprising A04184 (FIG. 4D) revealed a single species indicating no self-complementarity induced truncations and therefore an AAV viral vector with the single intended rAAV vector. This finding is supported by FIG. 4E which visualizes the sequencing coverage of the plasmid (pAAV vector) containing the genome for A04184. The sequencing reads almost completely map to the intended genome ITRs/expression cassette.

Evaluation of scAAV Having Promoter Repeats

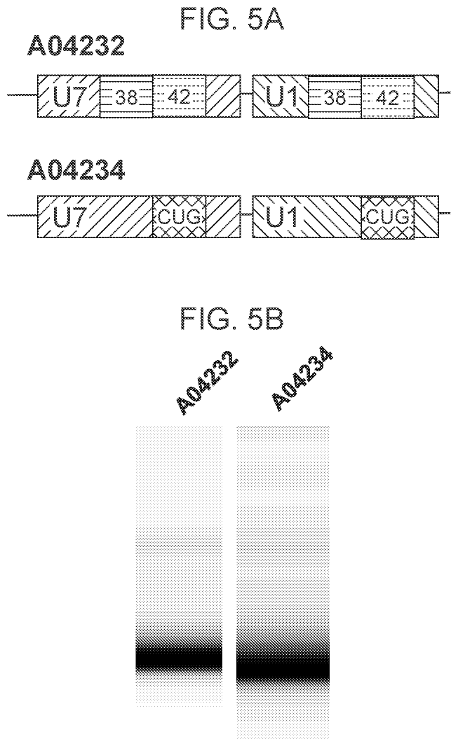

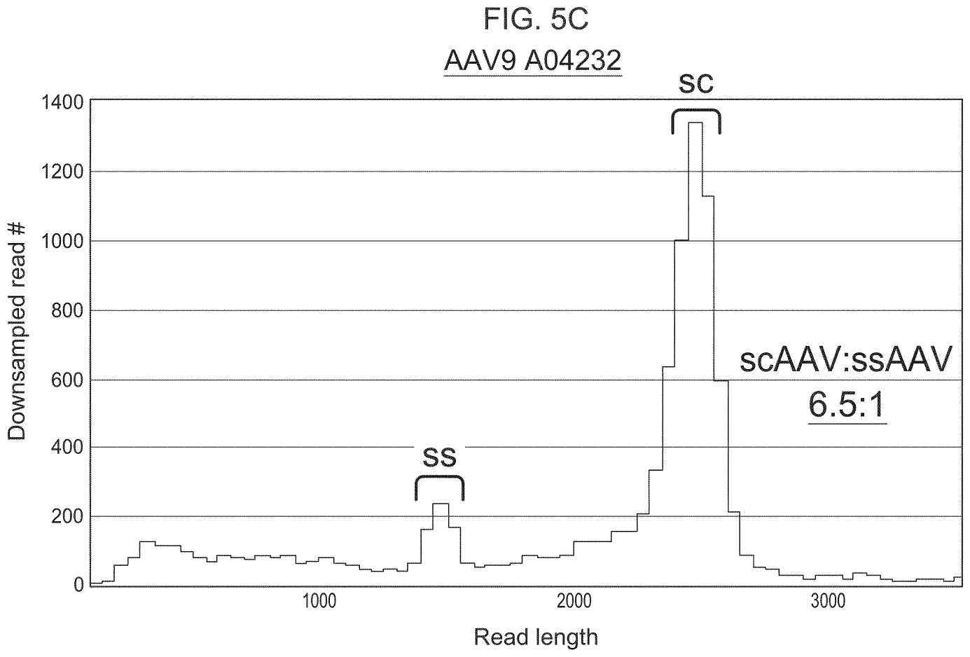

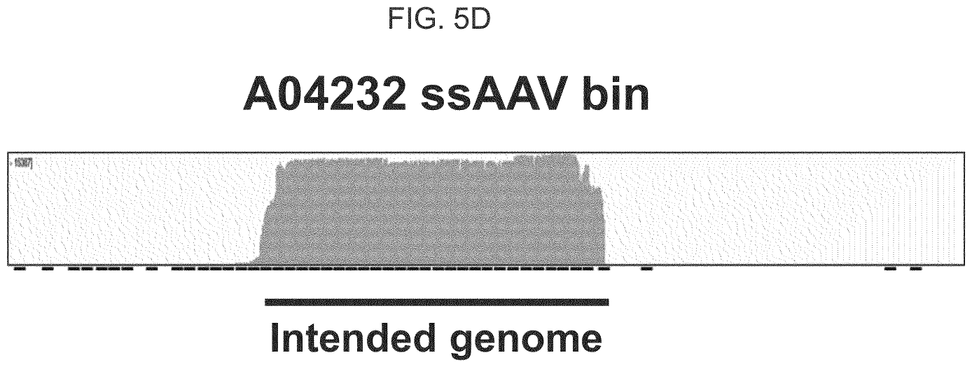

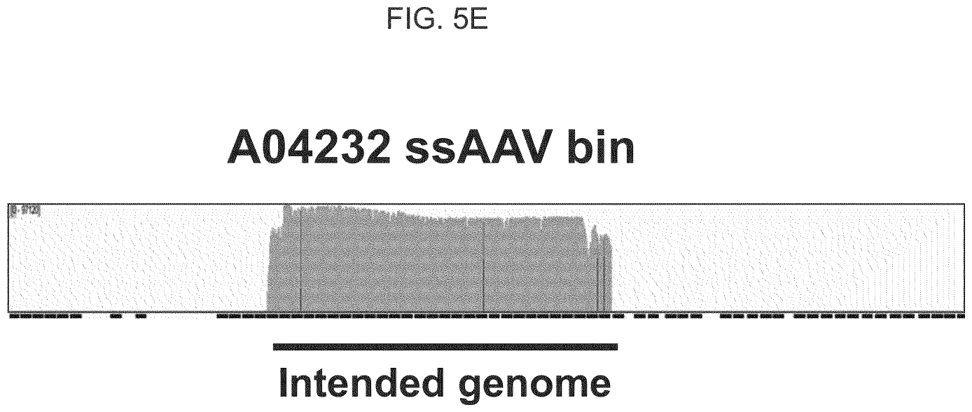

A04232 comprises snRNA 38/42 under the control of a U7 promoter and snRNA 38/42 under control of a U1 promoter (FIG. 5A). A04234 depicts an snRNA molecule targeting CUG repeats under the control of a U7 promoter and a second snRNA targeting CUG repeats under control of a U1 promoter (FIG. 5A).

Following packaging and purification of AAV viral vectors containing A04232 and A04234, Tapestation images reveal a predominant single species of each vector (FIG. 5B). Sequencing of an AAV viral vector comprising A04232 reveals a predominant species of the scAAV rAAV vector and smaller population of single stranded rAAV vector (FIG. 5C).

The sequencing coverage of the plasmid (pAAV vector) containing the genome for A04232 was visualized (FIG. 5D and FIG. 5E). The sequencing reads in the shorter “ssAAV bin” almost completely map to the intended single stranded genome ITRs/expression cassette. This indicates these are full length ssAAV genomes and not unintended partial genomes or truncations (FIG. 5D). The sequencing reads in the longer “scAAV bin” almost completely map to the intended genome ITRs/expression cassette (FIG. 5E)

Evaluation of rAAV Vectors Having 4 Different snRNA Promoters

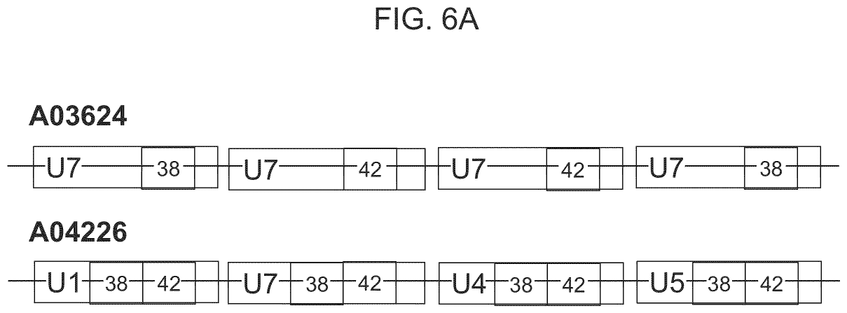

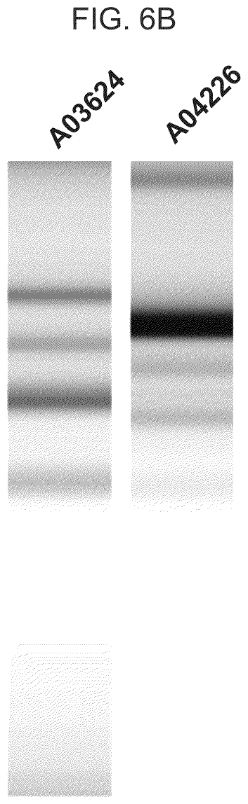

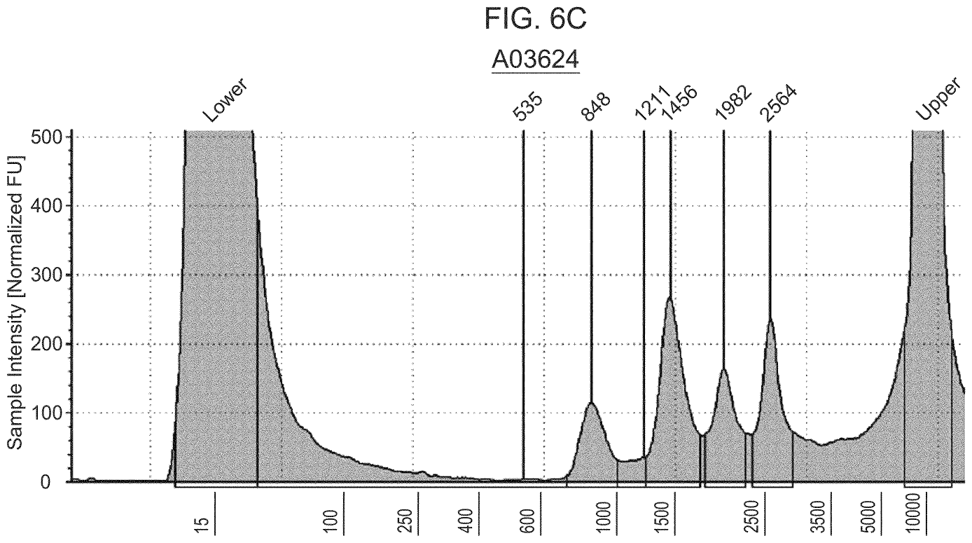

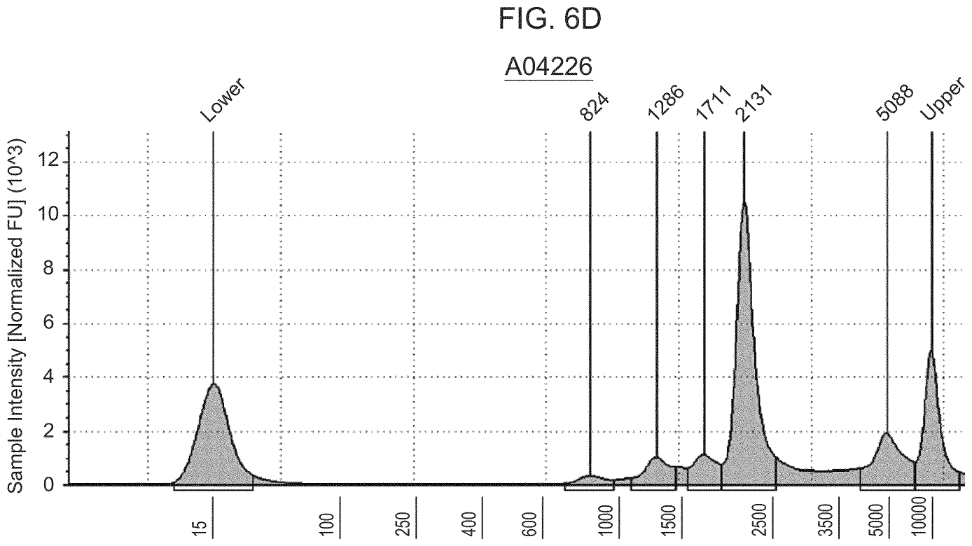

A03624 depicts a self-complementary AAV (scAAV) snRNA expression cassette comprising from 5′ to 3′: a U7 promoter driving expression of snRNA 38, a U7 promoter driving expression of snRNA 42, a U7 promoter driving expression of snRNA 42, and a U7 promoter driving expression of snRNA 38 (FIG. 6A). A04226 depicts from 5′ to 3′: a U1 promoter driving expression of snRNA 38/42, a U7 promoter driving expression of snRNA 38/42, a U4 promoter driving expression of snRNA 38/42, and a U5 promoter driving expression of snRNA 38/42 (FIG. 6A).

Following packaging and purification of AAV viral vectors containing A03624 and A04226, the packaging integrity of each was evaluated via Tapestation. The tapestation sample trace of the A03624 and A04226 is also depicted revealing multiple species (FIG. 6C and FIG. 6D). Vector A04226 reveals a dominant species.

snRNA Expression Cassettes Having Repeated and Varied Promoter Sequences

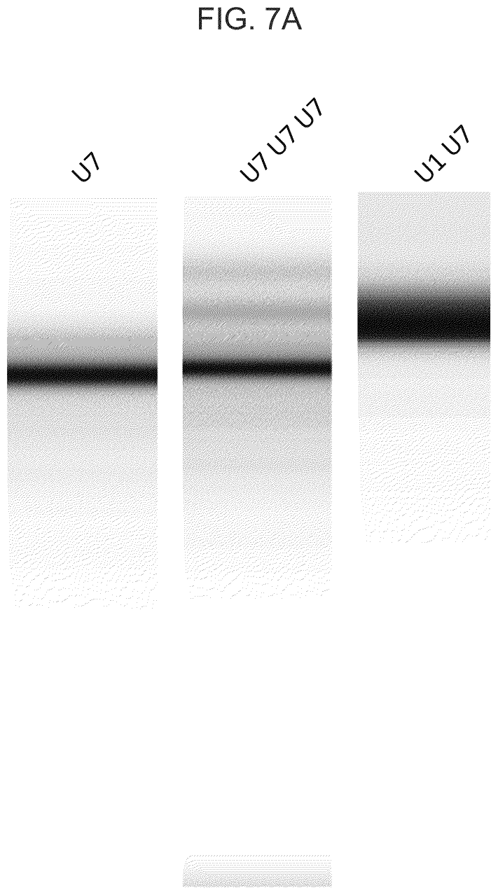

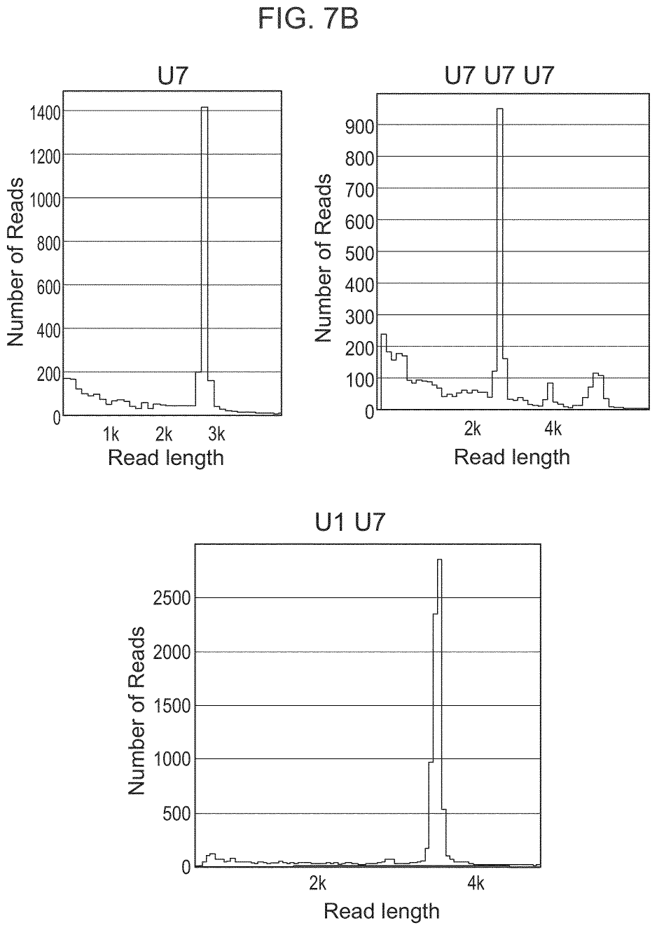

AAV viral vectors containing snRNA expression cassettes were constructed and the expression the packaged snRNA molecules was evaluated. Vectors were evaluated having one or more snRNA molecules, each under the control of a U7 promoter (FIG. 7A). Tapestation images revealed that a single U7 promoter driving expression of a single snRNA produced a single visible band. Triplicate snRNA regulated by U7 promoters produced multiple species evidenced by multiple bands. A dual U1 U7 promoter each regulating expression of an snRNA molecule produced a single band visible by Tapestation. Sequencing of each vector form FIG. 7A supported the tapestation observations (FIG. 7B).

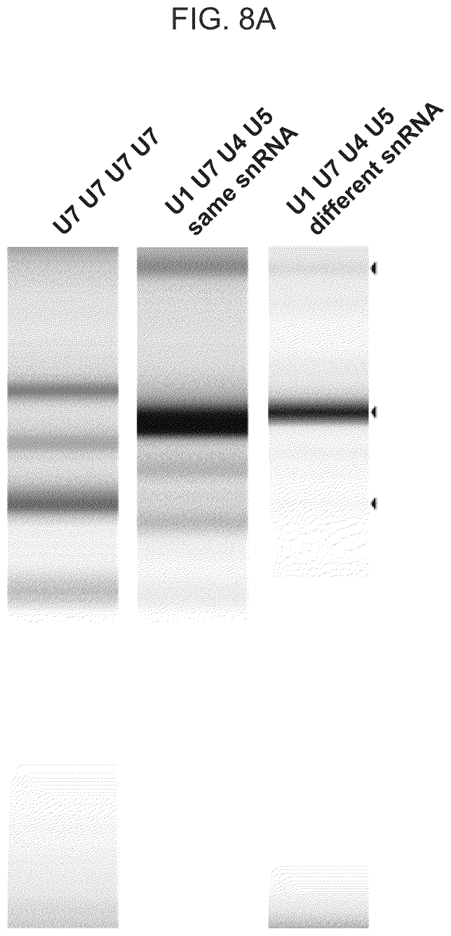

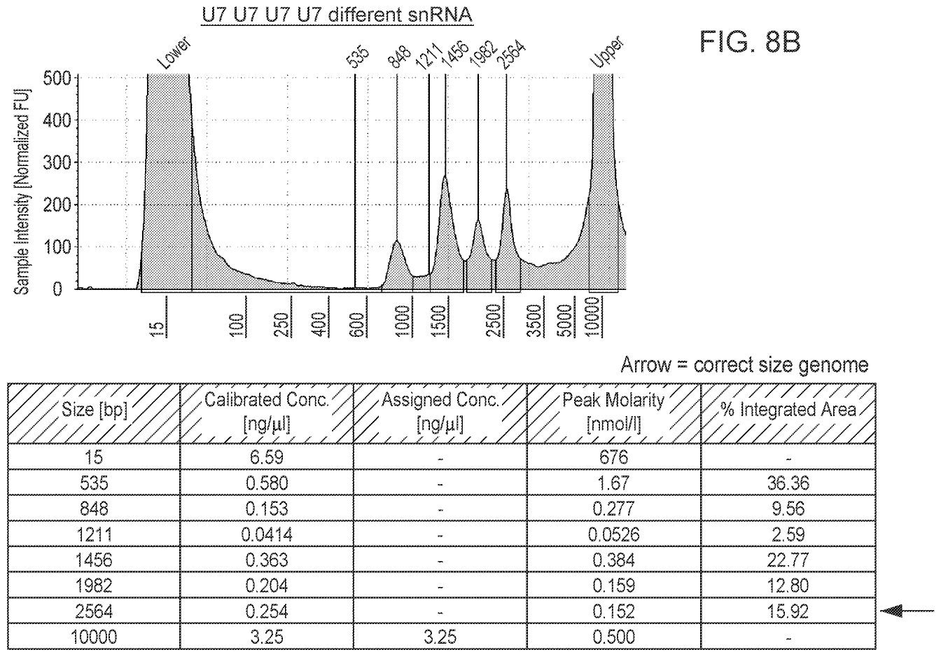

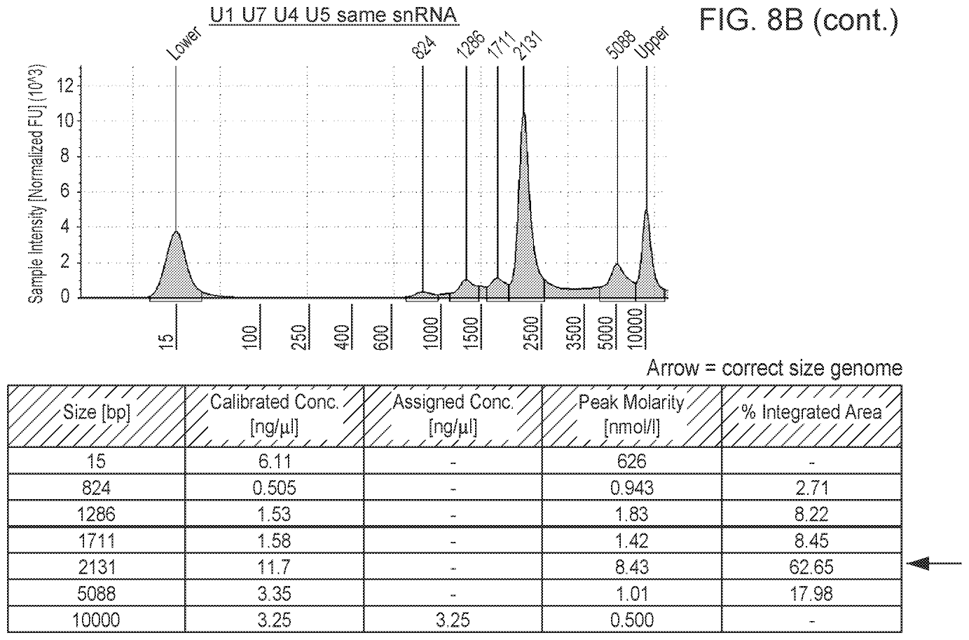

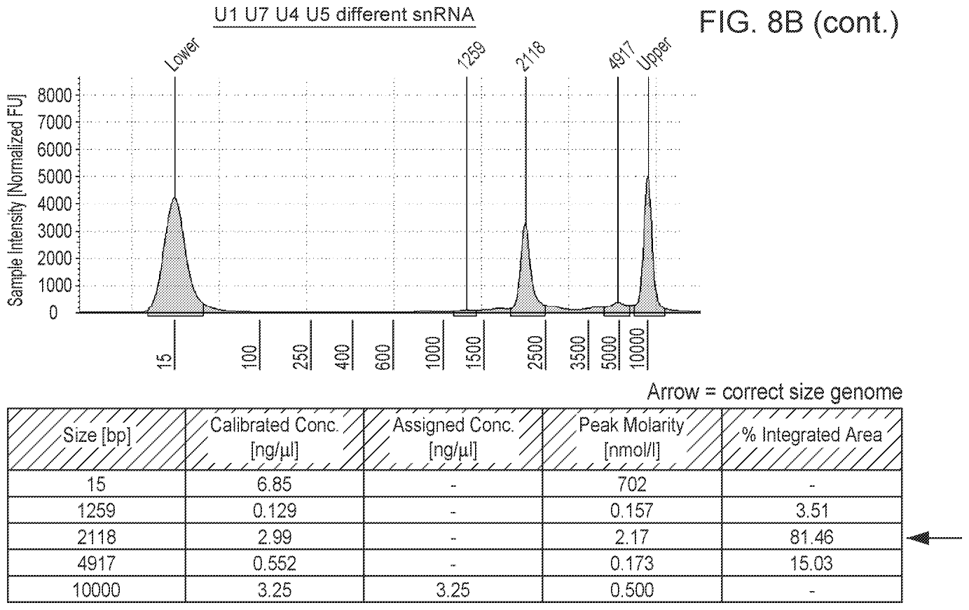

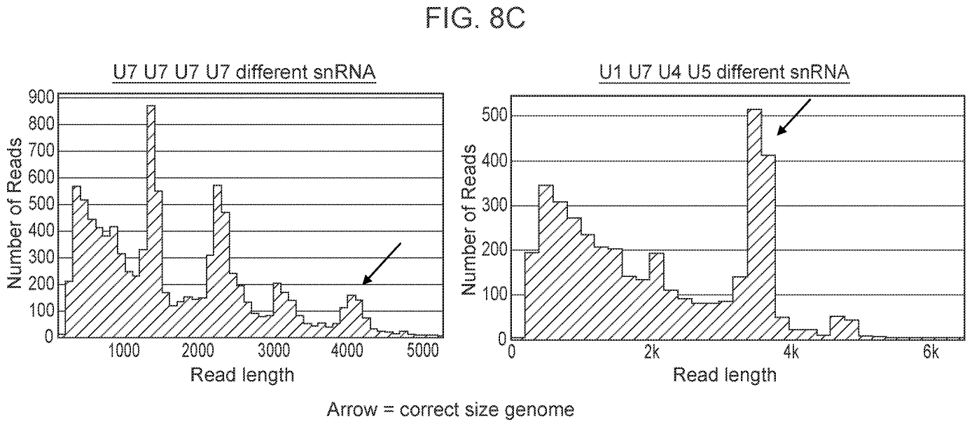

Further vectors were evaluated by Tapestation in FIG. 8A. “U7 U7 U7 U7” refers to an snRNA expression cassette wherein four U7 promoters each drive expression of at least one snRNA molecule. “U1 U7 U4 U5 same snRNA” refers to an snRNA expression cassette wherein a U1 promoter, a U7 promoter, a U4 promoter, and a U5 promoter each drive expression of a discrete copy of an snRNA molecule such that 4 copies of the snRNA molecule are present in the cassette. “U1 U7 U4 U5 different snRNA” refers to an snRNA expression cassette wherein a U1 promoter, a U7 promoter, a U4 promoter, and a U5 promoter each drive expression of a different snRNA molecule such that the expression cassette comprises 4 snRNA molecules having distinct sequences. The tapestation images and tapestation traces of FIGS. 8A and 8B demonstrate that the vectors lacking repeated promoter sequences produce significantly cleaner vector products with fewer truncation products i.e. greater genome integrity. This finding is supported by sequencing the AAV viral vector genomes (FIG. 8C).

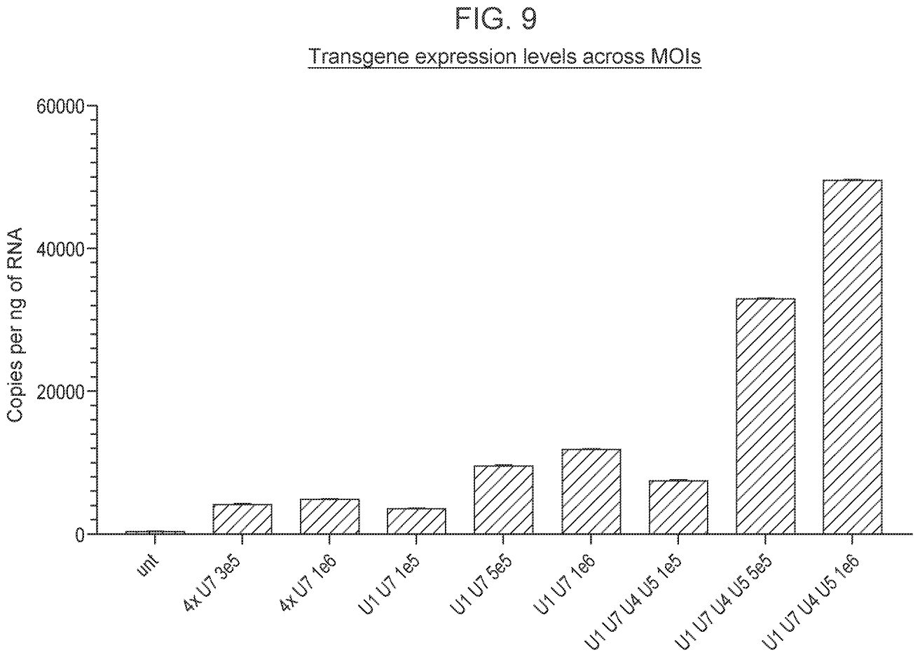

The expression levels of snRNA across a variety of multiplicities of infection (MOI) was evaluated for a variety of rAAV vectors having snRNA expression cassettes (FIG. 9). Evaluated snRNA expression cassettes include: a) 4×U7 (four U7 promoters each driving expression of the same snRNA molecule at two different MOIs 3e5 and 1e6; b) U1 and U7 promoters each driving expression of an snRNA molecule at three MOIs 1e5, 5e5, and 1e6; c) U1, U7, U4 and U5 promoters each driving expression of an snRNA molecule at three MOI2 1e5, 5e5, and 1e6. The cassettes having varied promoter sequences produced significantly greater copies numbers per ng of RNA.

snRNA Expression Cassettes Having Human and Murine Promoter Sequences

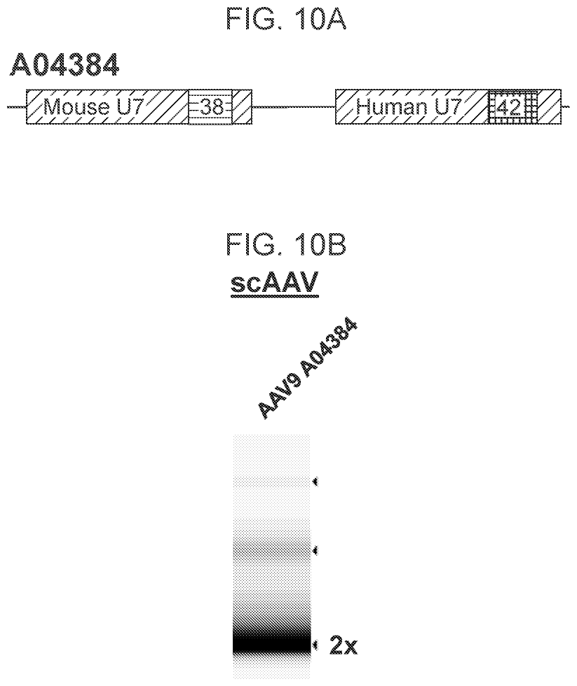

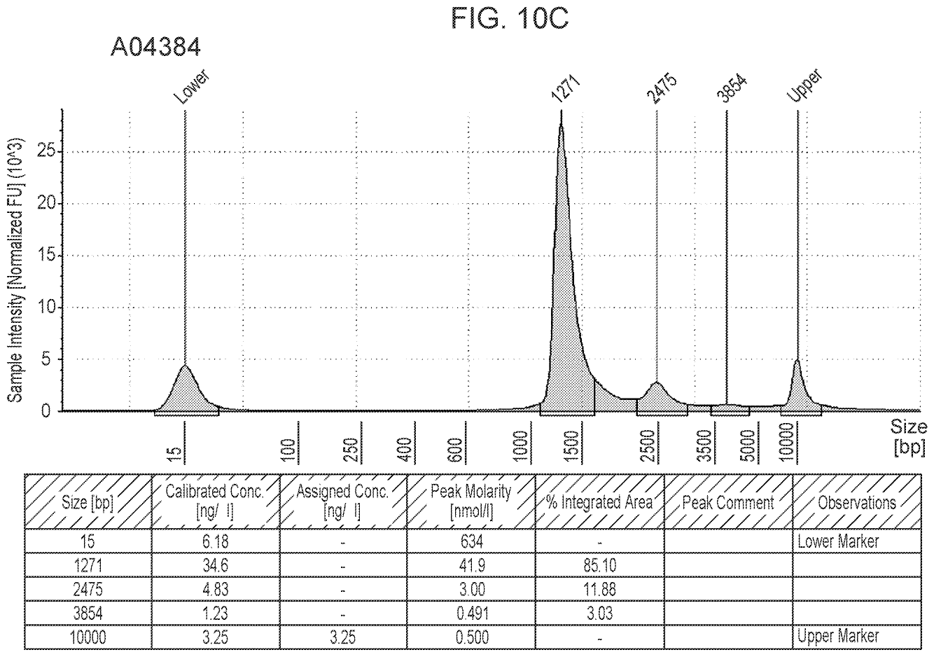

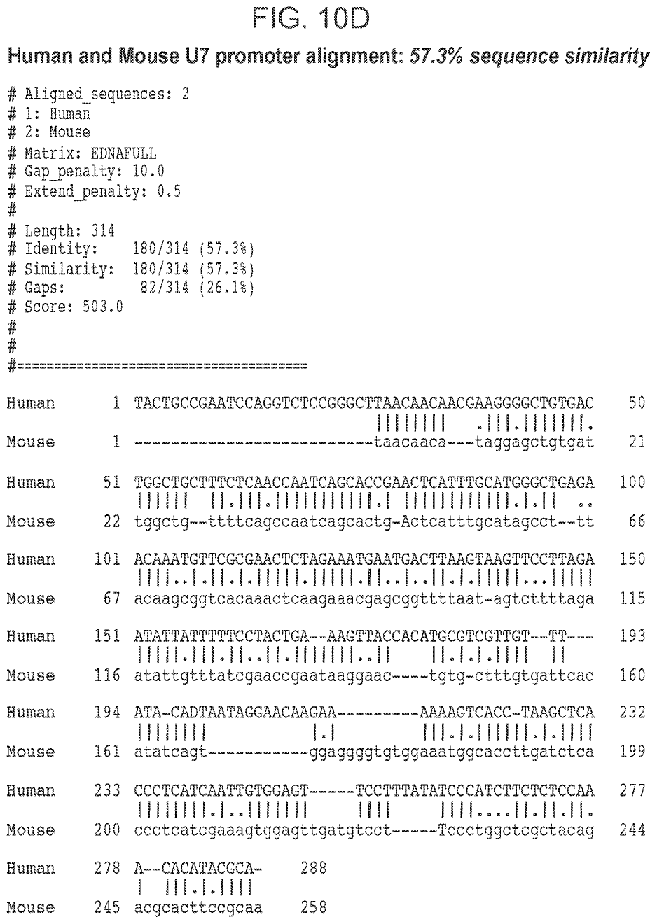

The use of related promoters human U7 and murine U7 were evaluated. The evaluated promoters have 57.3% identity and 57.3% similarity (FIG. 10D). A04384 depicts a scAAV snRNA expression cassette comprising snRNA 38 controlled by a murine U7 promoter and snRNA 42 controlled by a human U7 promoter (FIG. 10A). FIG. 10B is a tapestation images depicting rAAV vectors comprising the snRNA expression cassette of FIG. 10A. FIG. 10C is a tapestation trace of the tapestation image of FIG. 10B.

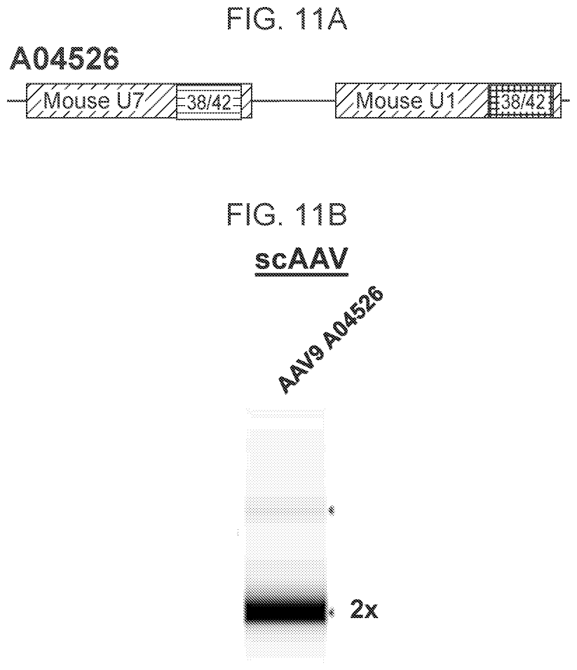

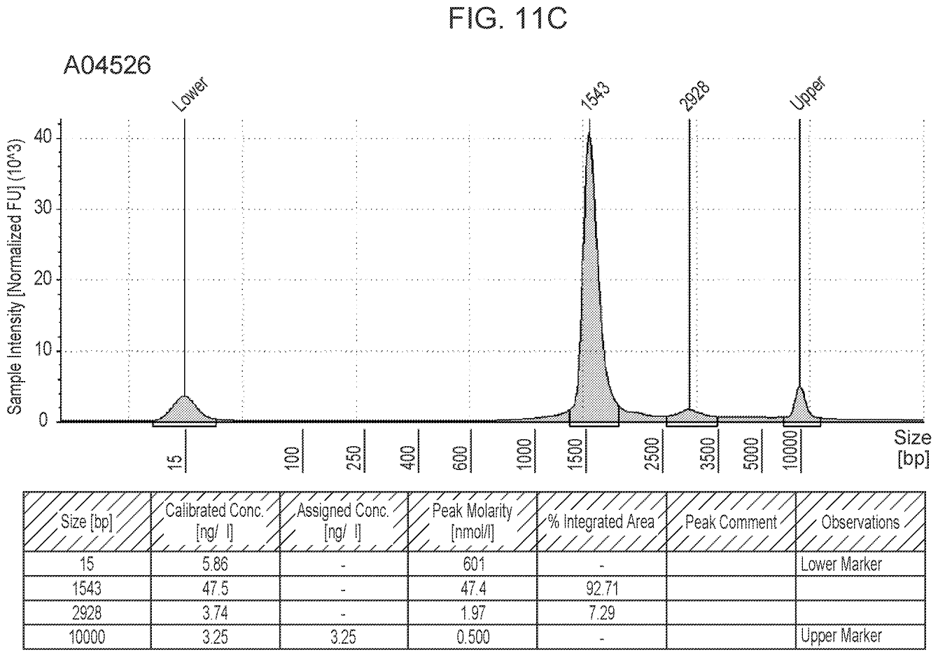

A04526 depicts a scAAV snRNA expression cassette comprising snRNA 38/42 controlled by a murine U7 promoter and snRNA 38/42 controlled by a human U7 promoter (FIG. 11A). FIG. 11B is a tapestation images depicting rAAV vectors comprising the snRNA expression cassettes of FIG. 11A. FIG. 11C is a tapestation trace of the tapestation image of FIG. 10B.

Analysis of the tapestation image and trace from A04526 and A04384 reveals that the desired rAAV vector is the dominant species. This indicates that murine and human U7 promoters do not have high enough identity to result in self-complementarity based truncations.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or embodimented herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

What is claimed is:

1. A recombinant Adeno-Associated Virus (rAAV) vector comprising:
a first inverted terminal repeat (ITR) sequence,
a first promoter sequence,
a first nucleic acid element,
a second promoter sequence,
a second nucleic acid element, and
a second ITR sequence,
wherein the first promoter sequence and second promoter sequence are distinct promoter sequences.

2. The rAAV vector of claim 1, wherein the first nucleic acid element and second nucleic acid element comprise a nucleic acid encoding a noncoding RNA or a transgene.

3. The rAAV vector of claim 1, wherein the noncoding RNA is a small-nuclear RNA (snRNA) molecule, a single guide RNA molecule (sgRNA), a microRNA, a short hairpin RNA (shRNA), an enhancer RNA (eRNA), a small nucleolar RNA (snoRNA) or a long noncoding RNA (lncRNA).

4. The rAAV vector of any one of the preceding claims, further comprising one or more additional promoter sequences.

5. The rAAV vector of any one of the preceding claims, wherein the one or more additional promoter sequences are distinct from the first promoter sequence and the second promoter sequence.

6. The rAAAV vector of any one of the preceding claims, further comprising one or more additional nucleic acid elements.

7. The rAAV vector of any one of the preceding claims, wherein the first promoter sequence has less than about 75% sequence identity to the second promoter sequence and the one or more additional promoter sequences.

8. The rAAV vector of any one of the preceding claims, wherein there are from about 50 to about 5,000 nucleotides between the 3′ end of the first promoter and the 5′ start of the second promoter.

9. The rAAV vector of any one of the preceding claims, wherein the length of each of the first nucleic acid elements, the second nucleic acid element, and the one or more additional nucleic acid elements is between about 50 and 5,000 nucleotides.

10. The rAAV vector of any one of the preceding claims, wherein the first nucleic acid element, second nucleic acid element, and/or one or more additional nucleic acid elements comprise one snRNA molecule.

11. The rAAV vector of any one of the preceding claims, wherein the first nucleic acid element, second nucleic acid element, and/or one or more additional nucleic acid elements comprises two snRNA molecules.

12. The rAAV vector of any one of the preceding claims, wherein the two snRNA molecules are separated by a spacer sequence site.

13. The rAAV vector of any one of the preceding claims, wherein the snRNA molecule is a modified snRNA molecule.

14. The rAAV vector of any one of the preceding claims, wherein the snRNA molecule comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 34-SEQ ID NO: 40.

15. The rAAV vector of any one of the preceding claims, wherein the promoter is selected from a U1, U2, U4, U5, U6, U7, H1, 7SK, or tRNA promoter.

16. The rAAV vector of any one of the preceding claims, wherein the promoter controls expression of an mRNA-encoding gene.

17. The rAAV vector of any one of the preceding claims, wherein the U1 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 3, SEQ ID NO: 4 or SEQ ID NO: 11.

18. The rAAV vector of any one of the preceding claims, wherein the U4 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 6.

19. The rAAV vector of any one of the preceding claims, wherein the U5 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 7 or SEQ ID NO: 14.

20. The rAAV vector of any one of the preceding claims, wherein the U7 promoter comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 1, SEQ ID NO: 2, SEQ ID NO: 12, or SEQ ID NO: 13.

21. The rAAV vector of any one of the preceding claims, wherein the first ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 50-SEQ ID NO: 55.

22. The rAAV vector of any one of the preceding claims, wherein the second ITR sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 50-SEQ ID NO: 55.

23. The rAAV vector of any one of the preceding claims, further comprising one or more terminator sequences.

24. The rAAV vector of any one of the preceding claims, wherein the terminator sequence is a U1, U2, U4, U5, U6, or U7 terminator sequence.

25. The rAAV vector of any one of the preceding claims, wherein the terminator sequence is a polyA sequence or a PolIII termination sequence.

26. The rAAV vector of any one of the preceding claims, wherein the U1 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 21.

27. The rAAV vector of any one of the preceding claims, wherein the U4 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 23.

28. The rAAV vector of any one of the preceding claims, wherein the U5 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 24.

29. The rAAV vector of any one of the preceding claims, wherein the U7 terminator sequence comprises, consists essentially of, or consists of a nucleic acid sequence at least 65%, 70%, 75%, 80%, 85%, 90%, 95%, 96%, 97%, 98%, 99% or 100% (or any percentage in between) identical to SEQ ID NO: 20.

30. The rAAV vector of any one of the preceding claims, wherein the rAAV vector is a single-stranded AAV vector (ssAAV).

31. The rAAV vector of any one of the preceding claims, wherein the rAAV vector is a self-complementary AAV vector (scAAV).

32. An AAV viral vector comprising the rAAV vector of any one of the preceding claims wherein the viral vector comprises an AAV capsid protein.

33. The AAV viral vector of claim 32, wherein the AAV capsid protein is an AAV1 capsid protein, an AAV2 capsid protein, an AAV3 capsid protein, an AAV3B capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein, an AAVrh10 capsid protein, or a modified AAV capsid protein.

34. The AAV viral vector of claim 32, wherein the AAV viral vector exhibits greater expression in a subject or cell relative to an AAV viral vector comprising an rAAV vector comprising a single noncoding RNA or transgene or an rAAV vector comprising repeated promoter sequences operably linked to noncoding RNA molecules or transgene sequences.

35. A pharmaceutical composition comprising the AAV viral vector of claim 32.

36. A cell comprising the rAAV vector of claim 1 or the AAV viral vector of claim 32.

37. A method of treating a disease or disorder in a subject in need thereof comprising administering a therapeutically effective amount of the AAV viral vector of claim 29 or the pharmaceutical composition of claim 32.
